(19) United States
(12) Patent Application Publication
(10) Pub. No.: US 2025/0319245 A1
(43) Pub. Date: Oct. 16, 2025

(54) MONITORING AND EXTENDING PERFORMANCE OF IMPLANTABLE PIEZOELECTRIC-OPERATED PUMP BASED ON VOLTAGE AND CURRENT MEASUREMENTS

(71) Applicant: Boston Scientific Scimed, Inc., Maple Grove, MN (US)

(72) Inventors: Brian Thomas Swanson, Fridley, MN (US); Keith R. Maile, New Brighton, MN (US); Kevin James Ivanca, Minneapolis, MN (US)

(21) Appl. No.: 19/171,975

(22) Filed: Apr. 7, 2025

Related U.S. Application Data
(60) Provisional application No. 63/632,286, filed on Apr. 10, 2024.

(51) Int. Cl. A61M 5/142

(52) U.S. Cl. CPC A61M 5/14276; A61M 2205/70; A61M 2205/8206

(57) ABSTRACT

Operating an implanted fluid-operated device having a pump operated by a piezoelectric element includes: providing a waveform of electrical energy from an implanted battery to the piezoelectric element to drive the piezoelectric element to repeatedly change a volume of a fluid chamber defined between a base plate and a deformable diaphragm, the fluid chamber being in fluidic connection with the fluid reservoir and with the inflatable member, by deforming the deformable diaphragm to pump fluid from the fluid reservoir to the inflatable member; determining a first electrical parameter of the waveform of electrical energy provided to the piezoelectric element; comparing the determined first electrical parameter to a predetermined parameter; and based on the determined first electrical parameter, changing the electrical waveform provided to the piezoelectric element.

### CROSS-REFERENCE TO RELATED APPLICATION

This application claims priority to U.S. Provisional Patent Application No. 63/632,286, filed on Apr. 10, 2024, entitled “MONITORING AND EXTENDING PERFORMANCE OF IMPLANTABLE PIEZOELECTRIC-OPERATED PUMP BASED ON VOLTAGE AND CURRENT MEASUREMENTS”, the disclosure of which is incorporated by reference herein in its entirety.

### TECHNICAL FIELD

This disclosure relates generally to bodily implants, and more specifically to bodily implants including a fluid control system having one or more piezoelectric-operated pumps and/or valves.

### BACKGROUND

Active implantable fluid-operated inflatable devices can include one or more pumps that regulate the flow of fluid between different portions of the implantable device. One or more valves can be positioned within fluid passageways of the device to direct and control the flow of fluid to achieve inflation, deflation, pressurization, depressurization, activation, deactivation and the like of different fluid-filled components of the device. In some implantable fluid-operated devices, an implantable pumping device may be manually operated by the user to provide for the transfer of fluid between a reservoir and the fluid-filled implant components of the device. In some situations, manual operation of the pumping device may make it difficult to achieve consistent inflation, deflation, pressurization, depressurization, activation, deactivation and the like of the fluid-filled implant components. Inconsistent inflation, deflation, pressurization, depressurization, activation and/or deactivation of the fluid-filled implant device(s) may adversely affect patient comfort, efficacy of the device, and the overall patient experience. Some implantable fluid-operated devices include an electronic control system including an electronically controlled manifold providing for the transfer of fluid within the implantable fluid-operated device.

The use of the electronic control system may provide for more accurate actuation and control of the flow of fluid between components of the inflatable device, thus improving performance and efficacy of the device, as well as patient comfort and safety. The electronic control system may include one or more electronically-operated pumps and one or more valves to control the flow of fluid in the system, and the pumps and valves may be operated by way of piezoelectric elements associated with the pumps and valves. Electronically-operated pumps and valves are complex systems that have a number of modes of failure and performance degradation.

Thus, a need exists to monitor the performance of components of implantable devices having electronically-operated pumps and valves and to take corrective action in the event of a detected performance degradation.

### SUMMARY

According to a general aspect, an implantable fluid-operated device that is configured to control fluid flow between a fluid reservoir and an inflatable member includes: a battery configured for storing energy; a base plate; a deformable diaphragm; a fluid chamber defined between the base plate and the deformable diaphragm, the fluid chamber being in fluidic connection with the fluid reservoir and with the inflatable member. The base plate defines a first fluid passageway configured for providing fluid from the fluid reservoir into the fluid chamber and a second fluid passageway configured for providing fluid from the fluid chamber to the inflatable member. The implantable fluid-operated device further includes: a piezoelectric element attached to the deformable diaphragm; driver circuitry configured for providing a waveform of electrical energy from the battery to the piezoelectric element to drive the piezoelectric element to repeatedly change a volume of the fluid chamber by deforming the deformable diaphragm to pump fluid from the fluid reservoir to the inflatable member; and a first monitor circuit configured for determining a first electrical parameter of the waveform of electrical energy provided from the drive circuitry to the piezoelectric element to drive the piezoelectric element; and a processor configured to, based on the determined first electrical parameter, change the electrical waveform provided to the piezoelectric element.

In some implementations, the determined first electrical parameter includes a voltage of the waveform of electrical energy provided to the piezoelectric element.

In some implementations, the processor is configured to compare the determined voltage of the waveform of electrical energy provided to the piezoelectric element to a predetermined threshold voltage and, when the determined voltage exceeds the predetermined voltage, to change the electrical waveform provided to the piezoelectric element.

In some implementations, the determined first electrical parameter includes a rate of change of a voltage of the waveform of electrical energy provided to the piezoelectric element.

In some implementations, the processor is configured to compare the determined rate of change of the voltage of the waveform of electrical energy provided to the piezoelectric element to a predetermined threshold change of the voltage and, when the rate of change of the determined voltage exceeds the predetermined voltage, to change the electrical waveform provided to the piezoelectric element.

In some implementations, changing the electrical waveform provided to the piezoelectric element includes one or more of changing an amplitude of the waveform, changing a frequency of the waveform, and changing a rate of change of a voltage of the waveform with respect to time.

In some implementations, changing the electrical waveform provided to the piezoelectric element includes blocking the providing of the waveform to the piezoelectric element until a reset signal is received by the processor.

In some implementations, the determined first electrical parameter includes a current of the waveform of electrical energy provided to the piezoelectric element.

In some implementations, the processor is configured to compare the determined current of the waveform of electrical energy provided to the piezoelectric element to a predetermined threshold and, when the determined current exceeds the predetermined current, to change the electrical waveform provided to the piezoelectric element.

In some implementations, the implantable fluid-operated device further includes a second monitor circuit configured for determining a second electrical parameter of electrical energy provided from the battery to the driver circuitry and the processor is further configured to, based on the determined second electrical parameter, change the electrical waveform provided to the piezoelectric element.

In some implementations, implantable fluid-operated device further includes a fluid filter disposed within or at an end of the first fluid passageway or disposed within or at an end of the second fluid passageway, where the end of the first fluid passageway is distal from the fluid chamber and where the end of the second fluid passageway is distal from the fluid chamber.

In some implementations, the implantable fluid-operated device, further includes power transmission circuitry configured for receiving power from an external power source and providing power to charge the battery.

In another general aspect, a method of operating an implanted fluid-operated device having a pump operated by a piezoelectric element includes: providing a waveform of electrical energy from an implanted battery to the piezoelectric element to drive the piezoelectric element to repeatedly change a volume of a fluid chamber defined between a base plate and a deformable diaphragm, the fluid chamber being in fluidic connection with the fluid reservoir and with the inflatable member, by deforming the deformable diaphragm to pump fluid from the fluid reservoir to the inflatable member; determining a first electrical parameter of the waveform of electrical energy provided to the piezoelectric element; comparing the determined first electrical parameter to a predetermined parameter; and based on the determined first electrical parameter, changing the electrical waveform provided to the piezoelectric element.

In some implementations, the determined first electrical parameter includes a voltage of the waveform of electrical energy provided to the piezoelectric element, the method further including comparing the determined voltage of the waveform of electrical energy to a predetermined threshold voltage and, when the determined voltage exceeds the predetermined voltage, changing the electrical waveform provided to the piezoelectric element.

In some implementations, the determined first electrical parameter includes a rate of change of a voltage of the waveform of electrical energy provided to the piezoelectric element, the method further including comparing the determined rate of change of the voltage of the waveform of electrical energy provided to the piezoelectric element to a predetermined threshold change of the voltage and, when the rate of change of the determined voltage exceeds the predetermined voltage, changing the electrical waveform provided to the piezoelectric element.

In some implementations, changing the electrical waveform provided to the piezoelectric element includes one or more of changing an amplitude of the waveform, changing a frequency of the waveform, and changing a rate of change of a voltage of the waveform with respect to time.

In some implementations, changing the electrical waveform provided to the piezoelectric element includes blocking the providing of the waveform to the piezoelectric element until a reset signal is received.

In some implementations, the determined first electrical parameter includes a current of the waveform of electrical energy provided to the piezoelectric element.

In some implementations, the method further includes comparing the determined current of the waveform of electrical energy provided to the piezoelectric element to a predetermined threshold and, when the determined current exceeds the predetermined current, changing the electrical waveform provided to the piezoelectric element.

In some implementations, the method further includes determining a second electrical parameter of electrical energy provided from the battery; and based on the determined second electrical parameter, changing the electrical waveform provided to the piezoelectric element.

### BRIEF DESCRIPTION OF THE DRAWINGS

FIG. 1 is a block diagram of an implantable fluid-operated inflatable device.

FIG. 2 illustrates a system including an example implantable fluid-operated inflatable device.

FIG. 3A is a schematic diagram of a fluidic architecture of an implantable fluid-operated inflatable device.

FIG. 3B is a schematic diagram of a fluidic architecture of an implantable fluid-operated inflatable device.

FIG. 4A is an exploded view of an example valve device of a fluid control system of a fluid-operated inflatable device.

FIG. 4B is another exploded view of the example valve device shown in FIG. 4A.

FIG. 4C is a cross-sectional view of the example valve device shown in FIG. 4A, in a closed position.

FIG. 4D is a cross-sectional view of the example valve device shown in FIG. 4A, in an open position.

FIG. 5A is a schematic view of an example valve device including an example auxiliary flow control device, with the example valve device in an open position.

FIG. 5B is a schematic view of an example valve device including an example auxiliary flow control device, with the example valve device in a closed position.

FIG. 6A is an exploded view of an example pump device of a fluid control system of a fluid-operated inflatable device.

FIG. 6B is a cross-sectional view of the example pump device shown in FIG. 6A, in an open position.

FIGS. 7A, 7B, and 7C are cross-sectional views of example pump devices that includes a filter for capturing particulate matter in the fluid flow and/or for blocking the particulate matter from entering certain parts of the fluidic system (e.g., for blocking particulate matter from entering a pump chamber of the device).

FIG. 8 is a schematic end view of a filter foil.

FIGS. 9A, 9B, and 9C are cross-sectional views of example pump devices that includes a filter for capturing particulate matter in the fluid flow and/or for blocking the particulate matter from entering certain parts of the fluidic system (e.g., for blocking particulate matter from entering a pump chamber of the device).

FIG. 10 is a cross-sectional view of the valve device of FIG. 5A and 5B, but also including a filter located at an end of a second fluid passageway and a filter located within a first fluid passageway.

FIG. 11 is a schematic block diagram of a system for driving a piezoelectric element of a piezoelectric-operated pump or valve and for monitoring and controlling the performance of the piezoelectric element.

FIG. 12A is a graph of the voltage amplitude of an example waveform that can be a provided by the driver to the piezoelectric element to drive the piezoelectric element to cause a pump associated with the piezoelectric element to pump fluid.

FIG. 12B is a graph of the voltage amplitude of another example waveform that can be a provided by the driver to the piezoelectric element to drive the piezoelectric element to cause a pump associated with the piezoelectric element to pump fluid.

FIG. 12C is a graph of the voltage amplitude of another example waveform that can be a provided by the driver to the piezoelectric element to drive the piezoelectric element to cause a pump associated with the piezoelectric element to pump fluid.

FIG. 13A is a graph of the measured battery voltage for each therapeutic inflation of the inflatable member that occurs after the battery has been fully charged.

FIG. 13B is a graph of the measured current drawn from the battery during each therapeutic inflation of the inflatable member that occurs after the battery has been fully charged.

FIG. 14 is an example graph of a voltage on a piezoelectric element as a function of time after a square wave pulse is provided to the piezoelectric element.

FIG. 15 is an example graph of the complex impedance of the piezoelectric element 1114 as a function of frequency over a range of frequencies from 0 to 60 Hz.

FIG. 16A is a graph of an example normal waveform (solid line) having a peak-to-peak amplitude of about 100 V and a frequency of about 50 Hz that can be provided from the driver to the piezoelectric element when the piezoelectric element is in a new, undegraded condition.

FIG. 16B is a graph of an example normal waveform (solid line) having a peak-to-peak amplitude of about 100 V and a frequency of about 50 Hz that can be provided from the driver to the piezoelectric element when the fluidic components of the implantable device are in a normal operating state.

FIG. 16C is a graph of an example normal waveform (solid line) having a peak-to-peak amplitude of about 100 V and a frequency of about 50 Hz that can be provided from the driver to the piezoelectric element when the battery is in a new, undegraded condition.

FIG. 16D is a graph of an example square wave waveform having a peak-to-peak amplitude of about 100 V and a frequency of about 50 Hz that can be provided from the driver to the piezoelectric element when a fluidic component of the implantable device is determined to be degraded as a result of a restriction of the fluid flow in the implantable device.

FIG. 16E is a graph of an example trapezoid wave waveform having a peak-to-peak amplitude of about 100 V and a frequency of about 50 Hz that, like the square wave of FIG. 16D, can be provided from the driver to the piezoelectric element when a fluidic component of the implantable device is determined to be degraded as a result of a restriction of the fluid flow in the implantable device.

FIG. 17 is a flowchart of an example process for operating an implanted fluid-operated device having a piezoelectric-operated pump for controlling fluid flow between an implanted fluid reservoir and an implanted inflatable member.

### DETAILED DESCRIPTION

Detailed implementations are disclosed herein. However, it is understood that the disclosed implementations are merely examples, which may be embodied in various forms. Therefore, specific structural and functional details disclosed herein are not to be interpreted as limiting, but merely as a basis for the claims and as a representative basis for teaching one skilled in the art to variously employ the implementations in virtually any appropriately detailed structure. Further, the terms and phrases used herein are not intended to be limiting, but to provide an understandable description of the present disclosure.

The terms “a” or “an,” as used herein, are defined as one or more than one. The term “another,” as used herein, is defined as at least a second or more. The terms “including” and/or “having,” as used herein, are defined as comprising (i.e., open transition). The term “coupled” or “moveably coupled,” as used herein, is defined as connected, although not necessarily directly and mechanically.

In general, the implementations are directed to bodily implants. The term patient or user may hereinafter be used for a person who benefits from the medical device or the methods disclosed in the present disclosure. For example, the patient can be a person whose body is implanted with the medical device or the method disclosed for operating the medical device by the present disclosure.

An implantable fluid-operated inflatable device may include a fluid control system. In some examples, the fluid control system includes at least one pump and/or at least one valve and/or at least one combined pump and valve device. In some examples, the components of the fluid control system control the flow of fluid between a fluid reservoir and an inflatable member of the implantable fluid-operated inflatable device, to provide for the inflation/pressurization and deflation/depressurization of the inflatable member. In some implementations, the fluid control system can be electronically-operated.

For example, the pumps and/or valves of the fluid control system can be electronically-operated by the fluid control system to control the pressure of, and the flow of fluid in, parts of the fluid-operated inflatable device. An electronically-operated fluid control system, in accordance with implementations described herein, can include a plurality of electromechanical devices, such as, piezoelectric devices that operate the pumps valves in the system. One or more controllers can control the electromechanical devices. Additionally, the one or more controllers can monitor the performance and electrical properties of the electromechanical devices to detect errors, failures, and degradation of the devices. When an error, failure, or degradation of an errors, failures, and degradation of an electromechanical device is detected, the one or more controllers can adjust the electronic control of the electromechanical device to facilitate continued operation of the electromechanical device and the safety of the patient in whom the inflatable device is implanted.

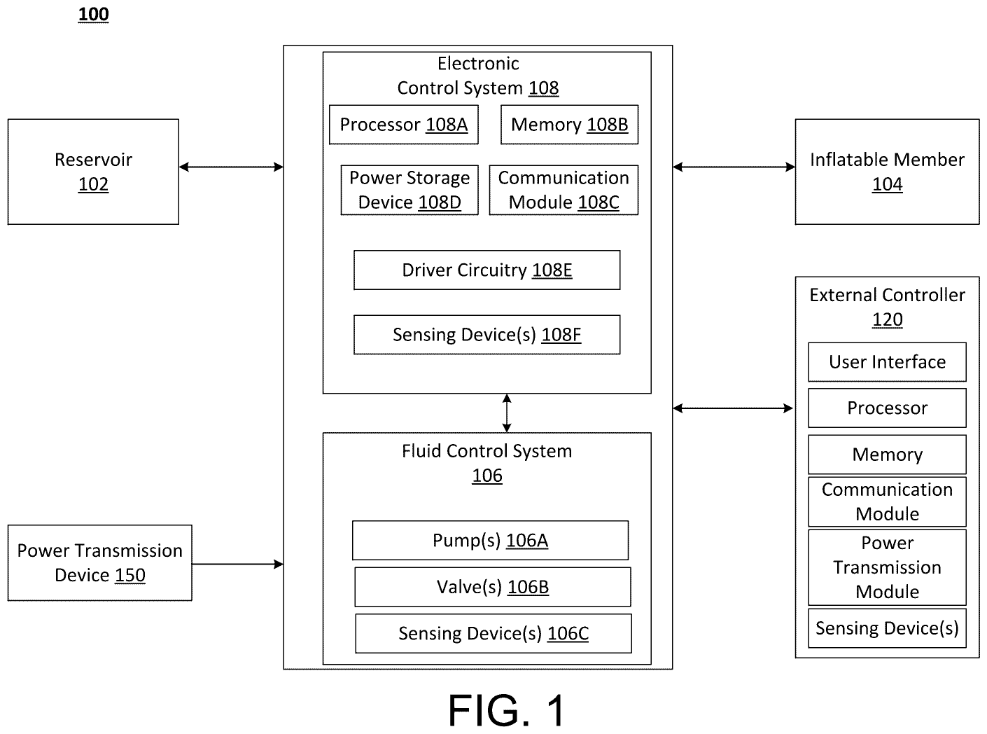

FIG. 1 is a block diagram of an example implantable fluid-operated inflatable device 100. The example inflatable device 100 shown in FIG. 1 includes a fluid reservoir 102, an inflatable member 104, and an electronic control system 108. The electronic control system 108 may interface with a fluid control system 106. The fluid control system 106 can include fluidics components such as one or more pumps 106A, one or more valves 106B and the like configured to transfer fluid between the fluid reservoir 102 and the inflatable member 104. The fluid control system 106 can include one or more sensing devices 106C that sense conditions such as, for example, fluid pressure, fluid flow rate and the like within the fluidics architecture of the inflatable device 100. In some implementations, the electronic control system 108 includes components that provide for the monitoring and/or control of the operation of various fluidics components of the fluid control system 106 and/or communication with one or more sensing device(s) within the implantable fluid-operated inflatable device 100 and/or communication with one or more external device(s). In some examples, the electronic control system 108 includes components such as a processor 108A, a memory 108B, a communication module 108C, a power storage device 108D (e.g., a battery), electronic driver circuity 108E, sensing devices 108F such as, for example, voltage measurement circuitry, current measurement circuitry, an accelerometer, power transmission circuitry 108G for receiving power from an external power source and providing power to charge the power storage device 108D, and other such components configured to provide for the monitoring, operation, and control of the implantable fluid-operated inflatable device 100. In some examples, the communication module 108C of the electronic control system 108 may provide for communication with one or more external devices such as, for example, an external controller 120.

In some examples, the external controller 120 includes components such as, for example, a user interface, a processor, a memory, a communication module, a power transmission module, and other such components providing for operation and control of the external controller 120 and communication with the electronic control system 108 of the inflatable device 100. For example, the memory may store instructions, applications and the like that are executable by the processor of the external controller 120. The external controller 120 may be configured to receive user inputs via, for example, the user interface, and to transmit the user inputs, for example, via the communication module, to the electronic control system 108 for processing, operation, and control of the inflatable device 100. Similarly, the electronic control system 108 may, via the respective communication modules, transmit operational information to the external controller 120. This may allow operational status of the inflatable device 100 to be provided, for example, through the user interface of the external controller 120, to the user, may allow diagnostics information to be provided to a physician, a technician, and the like.

In some examples, the power transmission module of the external controller 120 provides for charging of the components of the internal electronic control system 108. In some examples, transmission of power for the charging of the internal electronic control system 108 can be, alternatively or additionally, provided by an external power transmission device 150 that is separate from the external controller 120. In some implementations the external controller 120 can include sensing devices such as one or more pressure sensors, one or more accelerometers, and other such sensing devices. In some implementations, a pressure sensor in the external controller 120 may provide, for example, a local atmospheric or working pressure to the internal electronic control system 108, to allow the inflatable device 100 to compensate for variations in pressure. In some implementations, an accelerometer in the external controller 120 may provide detected patient movement to the internal electronic control system 108 for control of the inflatable device 100.

The fluid reservoir 102, the inflatable member 104, the electronic control system 108 and the fluid control system 106 may be internally implanted into the body of the patient. In some implementations, the electronic control system 108 and the fluid control system 106 are coupled in, or incorporated into, a housing. In some implementations, at least a portion of the electronic control system 108 is physically separate from the fluid control system 106. In some implementations, some modules of the electronic control system 108 are coupled to, or incorporated into, the fluid control system 106, and some modules of the electronic control system 108 are separate from the fluid control system 106. For example, in some implementations, some modules of the electronic control system 108 are included in an external device (such as the external controller 120) that is in communication other modules of the electronic control system 108 included within the implantable fluid-operated inflatable device 100.

In some examples, electronic monitoring and control of the implantable fluid-operated inflatable device 100 may provide for improved patient control of the device, improved patient comfort, improved patient safety, and the like. In some examples, electronic monitoring and control of the implantable fluid-operated inflatable device 100 may afford the opportunity for tailoring of the operation of the inflatable device 100 by a physician without further surgical intervention. Fluidic architecture defining the flow and control of fluid through the implantable fluid-operated inflatable device 100, including the configuration and placement of fluidics components such as pumps, valves, sensing devices and the like, may allow the inflatable device 100 to precisely monitor and control operation of the inflatable device, effectively respond to user inputs, and quickly and effectively adapt to changing conditions both within the inflatable device 100 (changes in pressure, flow rate and the like) and external to the inflatable device 100 (pressure surges due to physical activity, impacts and the like, sustained pressure changes due to changes in atmospheric conditions, and other such changes in external conditions).

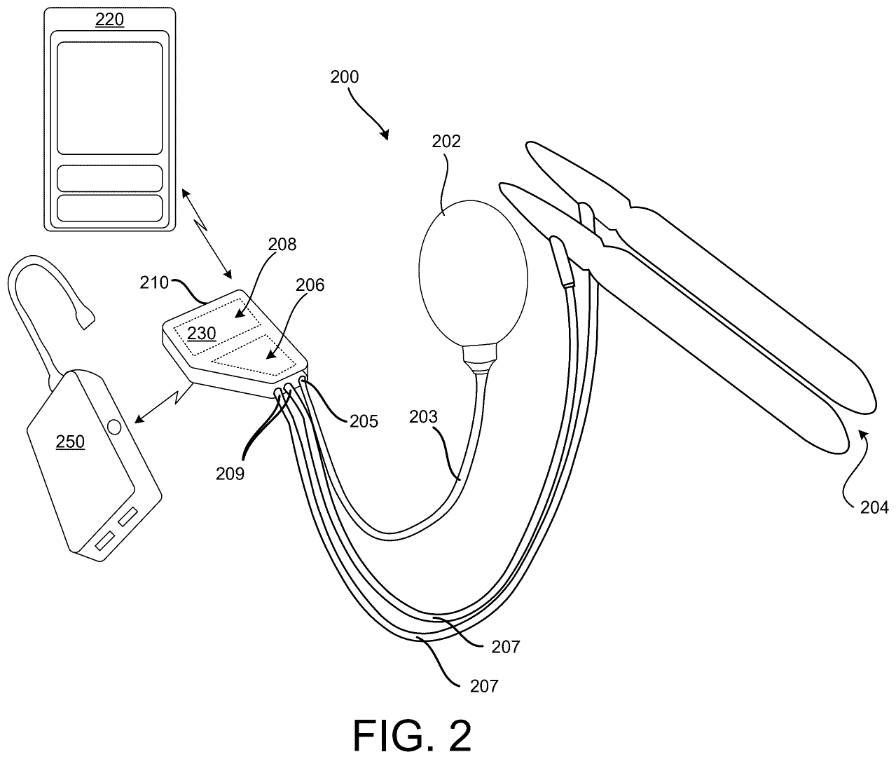

The example implantable fluid-operated inflatable device 100 may be representative of a number of different types of implantable fluid-operated devices. For example, the implantable fluid-operated inflatable device 100 shown in FIG. 1 may be representative of an inflatable penile prosthesis as shown in FIG. 2. In some implementations, the example implantable fluid-operated inflatable device 100 shown in FIG. 1 may be representative of other types of implantable inflatable devices that rely on the control of fluid flow to components of the device to achieve inflation, pressurization, deflation, depressurization, deactivation, and the like, such as, for example, an artificial urinary sphincter, and other such devices.

An example system including an example implantable fluid-operated inflatable device 200 in the form of an example inflatable penile prosthesis is shown in FIG. 2. The example inflatable device 200 includes a fluid control system 206 (similar to the example fluid control system 106 described above with respect to FIG. 1) including fluidics components such as pumps, valves, sensing devices and the like positioned in fluid passageways. In some implementations, the fluid control system includes components such as, for example, one or more fluid control devices, one or more pressure sensors, and other such components. In some implementations, the example inflatable device 200 includes an electronic control system 208 (similar to the example electronic control system 108 described above with respect to FIG. 1) configured to provide for the transfer of fluid between a reservoir 202 (such as the example fluid reservoir 102 described above with respect to FIG. 1) and an inflatable member 204 (similar to the example inflatable member 104 described above with respect to FIG. 1) via the fluidics components. In the example shown in FIG. 2, the inflatable member 204 is in the form of a pair of inflatable cylinders. In the example shown in FIG. 2, fluidics components of the fluid control system 206, and electronic components of the electronic control system 208 are received in a housing 210. In some implementations, fluidics components of the fluid control system 206, and electronic components of the electronic control system 208 received in the housing 210 together define an electronically controlled fluid manifold 230 that provides for the electronic control of the flow of fluid between the reservoir 202 and the inflatable member 204.

In the example shown in FIG. 2, a first conduit 203 connects a first fluid port 205 of the electronically controlled fluid manifold 230 (the fluid control system 206/electronic control system 208 received in the housing 210) with the reservoir 202. One or more second conduits 207 connect one or more second fluid ports 209 of the electronically controlled fluid manifold 230 (the fluid control system 206/electronic control system 208 received in the housing 210) with the inflatable member 204 in the form of the inflatable cylinders. In some examples, the electronic control system 208 can communicate with an external controller 220 (similar to the external controller 120 described above with respect to FIG. 1), via respective communication modules. For example, an application stored in a memory and executed by a processor of the external controller 220 may allow the user and/or a physician to operate, view, monitor and alter operation of the inflatable device 200. In some examples, components of the electronic control system 208 and/or the fluid control system 206 can be charged and/or recharged by a power transmission module of the external controller 220, and/or by a power transmission device 250, that is separate from the external controller 220.

The principles to be described herein are applicable to the example implantable fluid-operated inflatable device, in the form of the example inflatable penile prostheses shown in FIG. 2, and to other types of implantable fluid-operated inflatable devices that rely on pumps, valves and/or various fluidics components to provide for the transfer of fluid between the different fluid-filled implantable components to achieve inflation, deflation, pressurization, depressurization, deactivation, occlusion, and the like for effective operation. The example implantable fluid-operated inflatable device 200 shown in FIG. 2 includes an electronic control system 208 to provide for control of the operation of the respective inflatable members 204 in the form of cylinders, and the monitoring and control of pressure and/or fluid flow through inflatable members 204. Some of the principles to be described herein may also be applied to implantable fluid-operated inflatable devices that are manually controlled.

As noted above, the electronic control system 208 controlling the flow of fluid between the reservoir 202 and the inflatable member 204 for inflation, pressurization, deflation, depressurization and the like of the inflatable member 204 may provide for improved patient control of the inflatable device 200, improved accuracy in operation of the inflatable device 200, improved patient comfort, improved patient safety, and the like. In some situations, this improved control and improved accuracy in the operation of the inflatable device 200 may rely on precise operation and control of the components within the fluid control system 206 and/or the electronically controlled fluid manifold 230. Accordingly, in some implementations, the electronically controlled fluid manifold 230 includes a fluid control system 206 having one or more pump and/or valve devices. Accurate and consistent operation of the components of the pump and/or valve devices may produce the desired accurate flow control, and consistent inflation, deflation, pressurization, depressurization, deactivation, occlusion, and the like for effective operation.

A fluid control system, in accordance with implementations described herein, can include a pump assembly including, for example, one or more pump devices and valve devices and/or combined pump and valve devices within a fluid circuit of the pump assembly to control the transfer fluid between the fluid reservoir and the inflatable member. In some examples, the pump assembly including the one or more pump devices and valve device(s) and/or combined pump and valve devices is electronically controlled. In an example in which the pump assembly is electronically powered and/or controlled, the pump assembly may include a hermetic manifold that can contain and segment the flow of fluid from electronic components of the pump assembly, to prevent leakage and/or gas exchange. In some examples, the one or more pump devices and valve devices and/or combined pump and valve devices include electric elements that are configured to be electronically actuated to change their shape and thereby to function as a pump or valve. In some examples, the pump assembly includes one or more pressure sensing devices in the fluid circuit to provide for relatively precise monitoring and control of fluid flow and/or fluid pressure within the fluid circuit and/or the inflatable member. A fluid circuit configured in this manner may facilitate the proper inflation, deflation, pressurization, depressurization, and deactivation of the components of the implantable fluid-operated device to provide for patient safety and device efficacy.

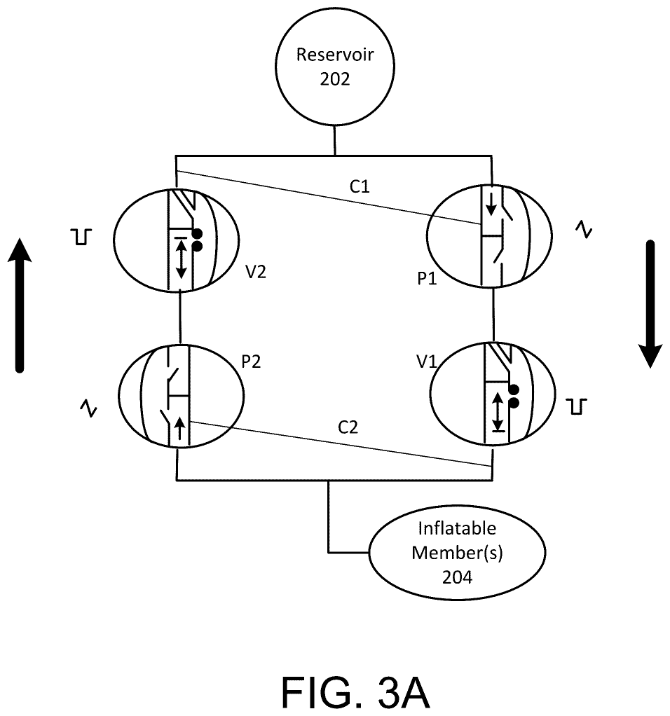

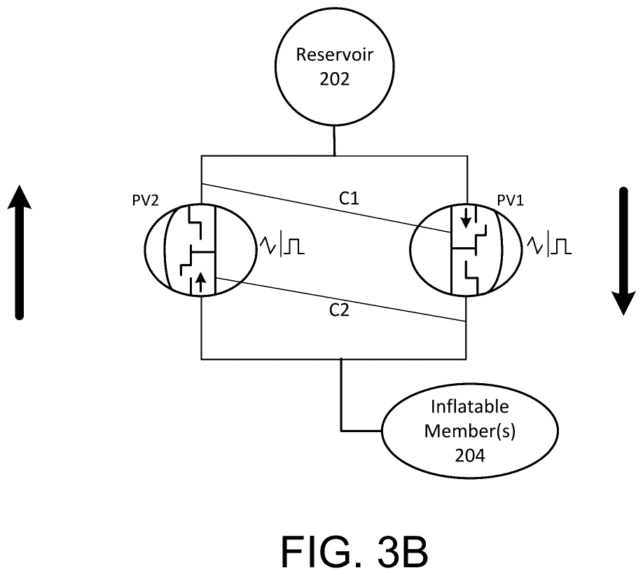

FIG. 3A is a schematic diagram of an example fluidic architecture for an electronically-operated implantable fluid-operated inflatable device, according to an aspect. FIG. 3B is a schematic diagram of an example fluidic architecture for an electronically-operated implantable fluid-operated inflatable device, according to an aspect. The fluidic architecture of an implantable fluid-operated inflatable device can include other arrangements of fluidic passageways, pump(s)/valve(s), pressure sensor(s) and other components than the examples shown in FIGS. 3A and 3B.

The example fluidic architecture shown in FIG. 3A includes a first pump P1 and a first valve V1 positioned in a first fluid passageway, between the reservoir 202 and the inflatable member 204, to control the flow of fluid from the reservoir 202 to the inflatable member 204. The example fluidic architecture shown in FIG. 3A includes a second pump P2 and a second valve V2 positioned in a second fluid passageway, between the inflatable member 204 and the reservoir 202, to control the flow of fluid from the inflatable member 204 to the reservoir 202. As shown in FIG. 3B, in some examples, the first pump and the first valve are included in a combination pump and valve device PV1 provided in the first fluid passageway, and the second pump and the second valve are included in a second combination pump and valve device PV2 provided in the second fluid passageway.

In example fluidic architecture shown in FIG. 3A, the first pump P1 and the first valve V1 operate to pump fluid from the reservoir 202 to the inflatable member 204 through the first fluid passageway to provide for inflation of the inflatable member 204, while the second valve V2 closes the second fluid passageway to prevent backflow of fluid, back to the reservoir 202. The second pump P2 and the second valve V2 operate to pump fluid from the inflatable member 204 to the reservoir 202 through the second fluid passageway to provide for deflation of the inflatable member 204, while the first valve V1 closes the first fluid passageway to prevent backflow of fluid to the inflatable member 204.

In an optional example implementation, a conduit C1 can connect a section of the second fluid passageway that is downstream of pump P2 and valve V2 to a section of the first fluid passageway, for example, to an inlet portion of pump P1. Fluid flow through conduit C1 can flush fluid and material from out of the section of the first fluid passageway when fluid is pumped from the inflatable member 204 to the reservoir 202. In an optional example implementation, a conduit C2 can connect a section of the first fluid passageway that is downstream of pump P1 and valve V1 to a section of the second fluid passageway, for example, to an inlet portion of pump P2. Fluid flow through conduit C2 can flush fluid and material from out of the section of the second fluid passageway when fluid is pumped from the reservoir 202 to the inflatable member 204.

In the example arrangement shown in FIG. 3B, the first combined pump and valve device PV1 and the second combined pump and valve device PV2 may be operated in a first mode to inflate or pressurize the inflatable member 204 and in a second mode to deflate or depressurize the inflatable member 204. In the first mode of operation, the first combined pump and valve device PV1 convey fluid from the reservoir 202 to the inflatable member 204, while the second combined pump and valve device PV2 remains closed/inoperable to prevent flow of fluid from the inflatable member 204 towards the reservoir 202 to prevent deflation/depressurization. The first combined pump and valve device PV1 may remain operable to pump fluid to the inflatable member 204 until a desired pressure is achieved. The first combined pump and valve device PV1 may be closed once the desired pressure is achieved, to maintain the inflatable member 204 at the desired pressure/inflated state. In the second mode of operation, the second combined pump and valve device PV2 convey fluid from the inflatable member 204 to the reservoir 202, while the first combined pump and valve device PV1 remains closed/inoperable to prevent flow of fluid from the reservoir 202 towards the inflatable member 204 to prevent inflation/pressurization. The second combined pump and valve device PV2 may remain operable to pump fluid to the reservoir 202 until a desired pressure is achieved at the inflatable member 204. The second combined pump and valve device PV2 may be closed once the desired pressure is achieved, to maintain the inflatable member 204 at the desired pressure/in the deflated state.

In an optional example implementation, a conduit C1 can connect a section of the second fluid passageway that is downstream of pump P2 and valve V2 to a section of the first fluid passageway, for example, to an inlet portion of pump P1. Fluid flow through conduit C1 can flush fluid and material from out of the section of the first fluid passageway when fluid is pumped from the inflatable member 204 to the reservoir 202. In an optional example implementation, a conduit C2 can connect a section of the first fluid passageway that is downstream of pump P1 and valve V1 to a section of the second fluid passageway, for example, to an inlet portion of pump P2. Fluid flow through conduit C2 can flush fluid and material from out of the section of the second fluid passageway when fluid is pumped from the reservoir 202 to the inflatable member 204.

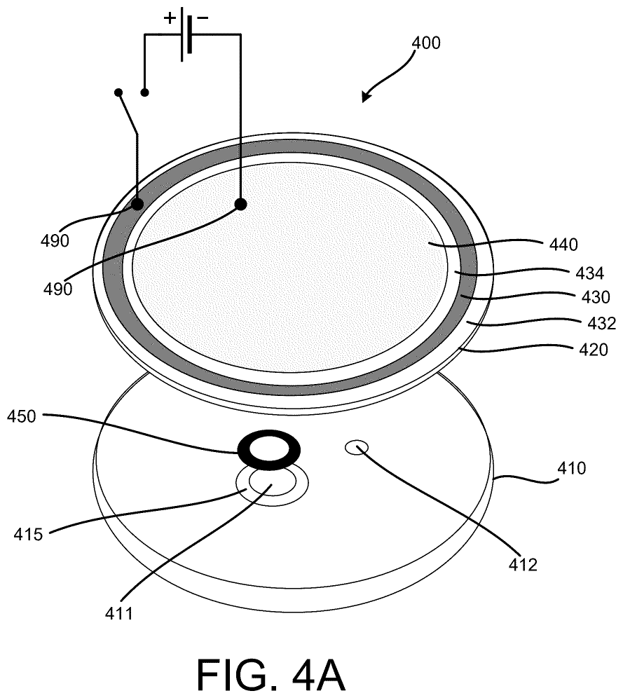

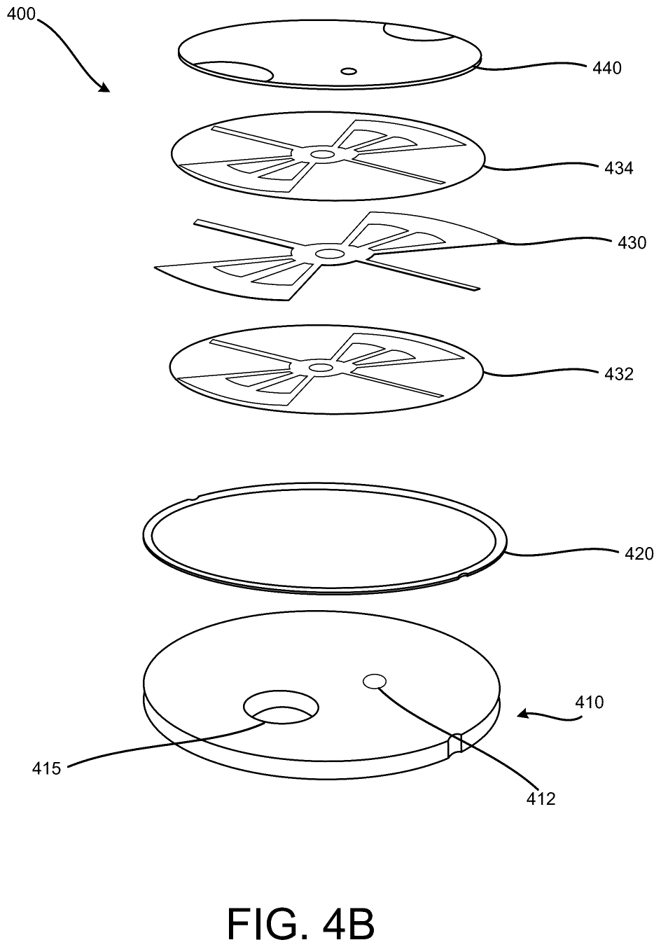

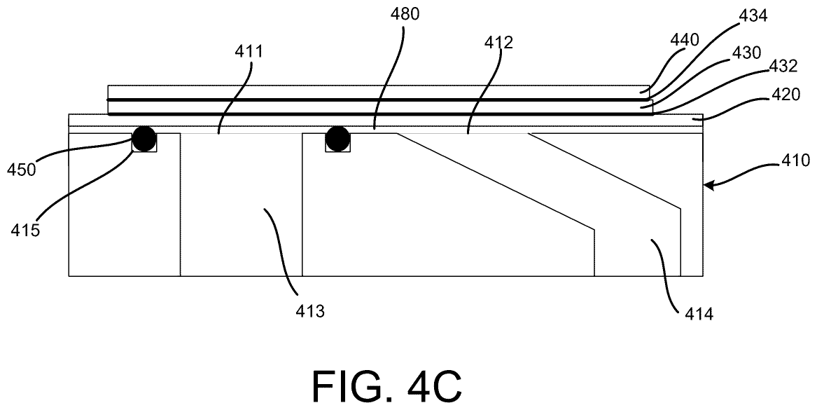

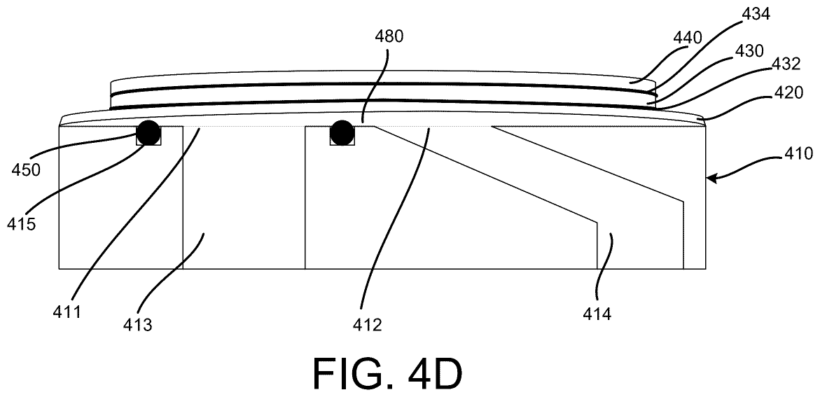

FIG. 4A is a partially exploded perspective view of an example valve device 400. FIG. 4B is an exploded perspective view of the example valve device 400. FIGS. 4C and 4D are cross-sectional views of the example valve device 400 shown in FIG. 4A, in an assembled state. The example valve device 400 shown in FIGS. 4A-4D is an example of a fluid control device, or a fluidic component, included in the fluid control system 206 of the example electronically controlled fluid manifold 230 described above.

In the example arrangement shown in FIGS. 4A-4D, the example valve device 400 includes a base plate 410 defining a base portion of the valve device 400. A diaphragm 420 is positioned on the base plate 410. A piezoelectric element 440 is positioned on the diaphragm 420, with an isolation layer 430 positioned between the diaphragm 420 and the piezoelectric element 440. The piezoelectric element can be electrically powered (e.g., by a battery of in the implantable fluid-operated inflatable device 100) to drive the diaphragm 420 to open and close the valve device 400. The diaphragm 420 can include a thin metal foil, whose shape can be repeatably deformed in response to movement by the piezoelectric element 440. In some implementations, the diaphragm 420 can include titanium material. In some implementations, the diaphragm 420 can include gold material. In some implementations, the diaphragm 420 can include stainless steel material or other alloys. In some implementations, the isolation layer 430 can include a polyamide material that has a high resistivity, for example, a resistivity greater than 1013 Ohm-cm to provide electrical isolation between the piezoelectric element 440 and the diaphragm 420.

In some examples, an epoxy layer 432 provides for the coupling of the isolation layer 430 and the diaphragm 420. In some examples, an epoxy layer 434 provides for the coupling of the piezoelectric element 440 and the isolation layer 430, and the epoxy layers 432, 434 together provide for the coupling of the piezoelectric element 440 to the diaphragm 420. In some implementations, the epoxy layers 432, 434 are not distinct but are part of one epoxy layer. The epoxy layers 432, 434 can be formed from a mixture of different chemicals (e.g., a resin and a hardener) that, when mixed and cured, react to form a covalent bond and that adhere to surfaces that they contact. Curing of the epoxy can be controlled through selection of the resin and hardener chemicals used in the mixture, selection of the ratio of the chemicals used in the mixture, control of the temperature of the mixture, and application of electromagnetic radiation to the mixture.

In some examples, one or more electrodes 490 are arranged on the example valve device 400. In the example shown in FIG. 4A, the example valve device 400 includes a pair of electrodes 490 coupled between the isolation layer 430 and the piezoelectric element 440. Application of a voltage to the piezoelectric element 440 causes a deflection or deformation of the piezoelectric element 440 and a corresponding deflection or deformation of the diaphragm 420 coupled thereto.

In the example arrangement shown in FIGS. 4A-4D, a fluid chamber 480 is defined between the base plate 410 and the diaphragm 420. For example, in some implementations, the diaphragm 420 can be bonded to the base plate 410 at the periphery of the diaphragm to form a fluid-tight connection between the base plate 410 and the diaphragm 420. The base plate 410 includes a first opening 411 that provides for communication between a first fluid passageway 413 and the fluid chamber 480. The base plate 410 includes a second opening 412 that provides for communication between a second fluid passageway 414 and the fluid chamber 480. In the example arrangement shown in FIGS. 4A-4D, the base plate 410 includes a recess 415 surrounding the first opening 411, with a seal 450, in the form of an O-ring in the example shown in FIGS. 4A-4D, fitted in the recess 415. In some examples, a top portion of the seal 450 is pressed against the diaphragm 420 in the closed position of the valve device 400, as shown in FIG. 4C to close off the chamber 480 and inhibit the flow of fluid through the example valve device 400, between the first fluid passageway 413 and the second fluid passageway 414 via the chamber 480. In some examples, in which the valve device 400 does not include a seal 450, the diaphragm 420 is seated against the base plate 410 to close off the chamber 480 and inhibit the flow of fluid through the valve device 400. In the open position of the example valve device 400, the base plate 410 and the top portion of the seal 450 are separated, or spaced apart from, the diaphragm 420 due to the deflection of the diaphragm 420. This positioning of the seal 450 and the base plate 410 relative to the diaphragm 420 opens the chamber 480 and allows fluid to flow through the example valve device 400, between the first fluid passageway 413 and the second fluid passageway 414 via the fluid chamber 480.

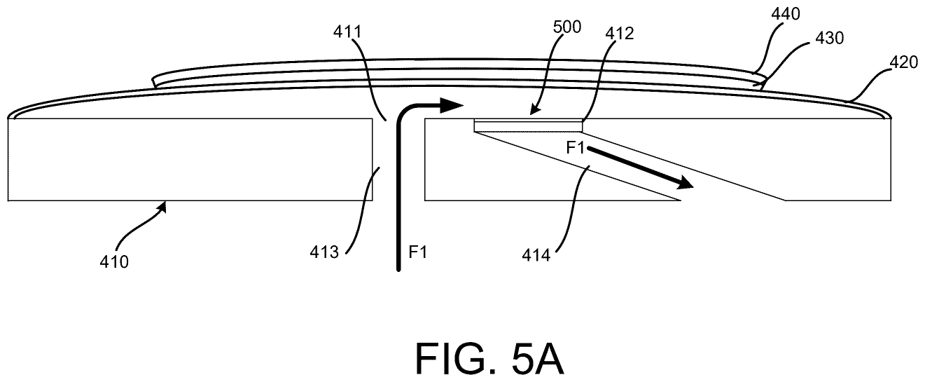

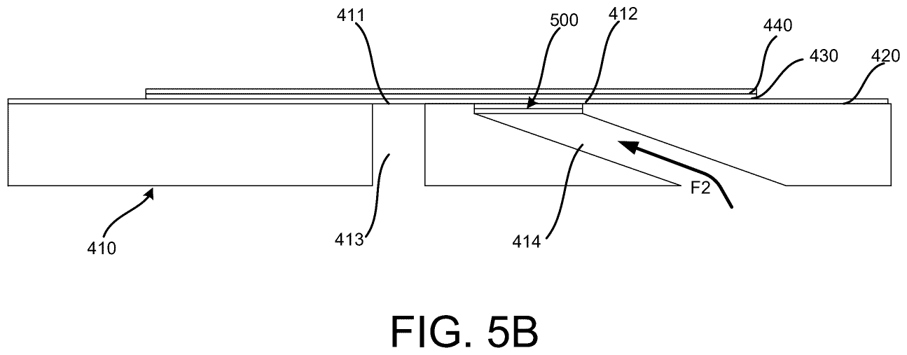

FIGS. 5A and 5B are cross-sectional views of the example valve device 400 shown in FIGS. 4A-4D, including an example flow control device 500 positioned in one of the fluid passageways of the example valve device 400.

FIG. 5A illustrates an example in which the valve device 400 is open, allowing fluid to flow in the direction of the arrows F1, through the first fluid passageway 413, into the chamber 480, and out of the valve device 400 through the second fluid passageway 414. The example shown in FIG. 5A may illustrate an open position of the valve device 400 that allows fluid to flow, for example, from the reservoir 202 to the inflatable member 204 to provide for inflation/pressurization of the inflatable member 204.

In the example arrangement shown in FIGS. 5A and 5B, the example flow control device 500 is positioned at the second opening 412 formed in the base plate 410, the second opening 412 providing for fluid communication between the fluid chamber 480 and the second fluid passageway 414. In some examples, the flow control device 500 is a check valve, or a one-way valve, that allows for flow in one direction (in this example, in the direction of the arrows F1), while inhibiting flow in the opposite direction.

FIG. 5B illustrates the closed position of the valve device 400, in which the flow of fluid through the valve device 400 is blocked. In some examples, the closed position shown in FIG. 5B may maintain an inflation pressure of the inflatable member 204. As described above, in some situations, pressure fluctuations and/or pressure spikes may exert a force, or pressure on the valve device 400 in the closed position. FIG. 5B illustrates a pressure spike, or a back pressure, exerted in the direction of the arrow F2. In the example described above with respect to FIGS. 4A-4D, this type of pressure spike, or back pressure exerted on the diaphragm 420/piezoelectric element 440 could cause an unintentional opening of the valve device 400, and an unintentional deflation/depressurization of the inflatable member 204. In the example shown in FIG. 5B, the flow control device 500 (positioned at the second opening 412, between the second fluid passageway 414 and the fluid chamber 480), for example, in the form of a check valve or a one-way valve, remains in the closed position in response to the pressure spike/back pressure/flow of fluid in the direction of the arrow F2. Thus, the positioning of the flow control device 500 at the second opening 412, allowing flow in a first direction, i.e., the direction of the arrows F1, while blocking flow in a second direction, i.e., the direction of the arrow F2, maintains the closed state of the valve device 400, even in response to fluctuation in pressure, or pressure spike, or back pressure.

The architecture and principles of operation of the valve device described above also can be used to implement one or more pumps (such as pumps that pumps P1, P2, PV1, PV2 of FIGS. 3A and 3B) to pump fluid from one location to another. For example, repeated movement of a diaphragm between an open position and a closed position, relative to a base plate, can cause fluid to be drawn into a chamber formed between the diaphragm and the base plate through a first fluid passageway and expelled out of the chamber into a second fluid passageway. In this manner, fluid can be pumped from a first location that is fluidically connected to the first passageway to a second location that is fluidically connected to the second passageway. In some implementations, one or more one-way valves can be configured to prevent, or limit, the flow of fluid in the direction from the second location to the first location.

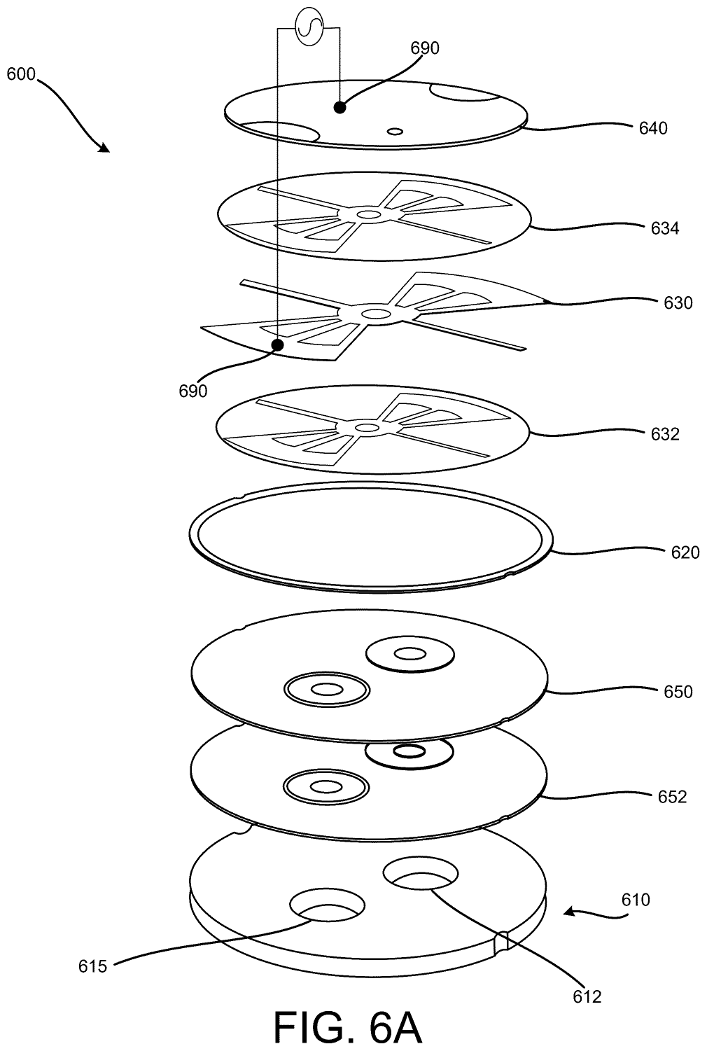

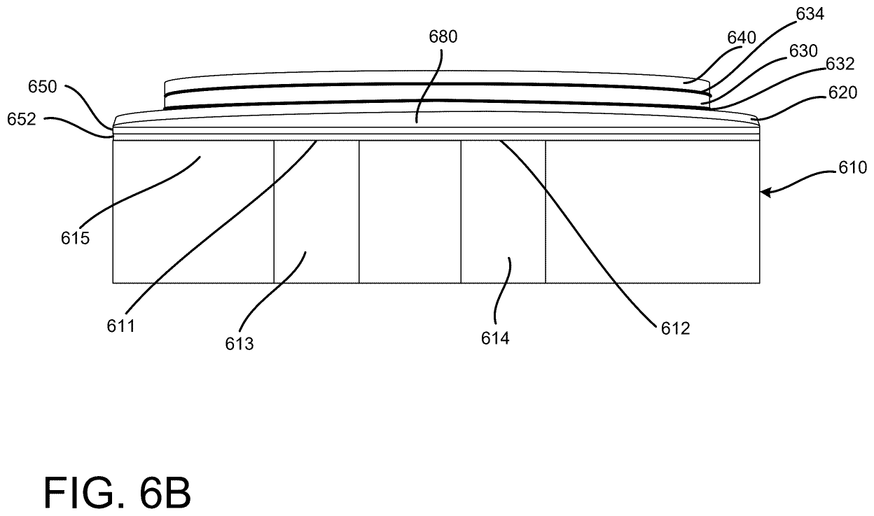

FIG. 6A is a partially exploded perspective view of an example pump device 600, and FIG. 6B is a cross-sectional view of the example pump device 600. The example pump device 600 shown in FIGS. 6A-6B is an example of a fluid control device, or a fluidic component, included in the fluid control system 206 of the example electronically controlled fluid manifold 230 described above.

In the example arrangement shown in FIGS. 6A-6B, the example pump device 600 includes a base plate 610 defining a base portion of the pump device 600. A diaphragm 620 is positioned on the base plate 610. A piezoelectric element 640 is positioned on the diaphragm 620, with an isolation layer 630 positioned between the diaphragm 620 and the piezoelectric element 640. The piezoelectric element can be electrically powered (e.g., by a battery of the implantable fluid-operated inflatable device 100) to drive the diaphragm 620 to pump fluid through the pump device 600. The diaphragm 620 can include a thin metal foil, whose shape can be repeatably deformed in response to movement by the piezoelectric element 640. In some implementations, the diaphragm 620 can include titanium material. In some implementations, the diaphragm 620 can include gold material. In some implementations, the diaphragm 620 can include stainless steel material or other alloys. In some implementations, the isolation layer 630 can include a polyamide material that has a high resistivity, for example, a resistivity greater than 10¹³ Ohm-cm to provide electrical isolation between the piezoelectric element 640 and the diaphragm 620.

In some examples, an epoxy layer 632 provides for the coupling of the isolation layer 630 and the diaphragm 620. In some examples, an epoxy layer 634 provides for the coupling of the piezoelectric element 640 and the isolation layer 630, and the epoxy layers 632, 634 together provide for the coupling of the piezoelectric element 640 to the diaphragm 620. In some implementations, the epoxy layers 632, 634 are not distinct but are part of one epoxy layer. The epoxy layers 632, 634 can be formed from a mixture of different chemicals (e.g., a resin and a hardener) that, when mixed and cured, react to form a covalent bond and that adhere to surfaces that they contact. Curing of the epoxy can be controlled through selection of the resin and hardener chemicals used in the mixture, selection of the ratio of the chemicals used in the mixture, control of the temperature of the mixture, and application of electromagnetic radiation to the mixture.

In some examples, one or more electrodes 690 are arranged on the example pump device 600. In the example shown in FIG. 6A, the example pump device 600 includes a pair of electrodes 690 coupled between the isolation layer 630 and the piezoelectric element 640. Application of a voltage to the piezoelectric element 640 causes a deflection or deformation of the piezoelectric element 640 and a corresponding deflection or deformation of the diaphragm 620 coupled thereto.

When the pump device 600 is used in the fluid control system 206 of the example electronically controlled fluid manifold 230 described above, the piezoelectric element 640 can be controlled to cause fluid to be pumped by device 600, for example, by repeatedly changing a volume of the fluid chamber 680 by deforming the deformable diaphragm 620 to pump fluid from the fluid reservoir to the inflatable member.

In the example arrangement shown in FIGS. 6A-6B, a fluid chamber 680 is defined between the base plate 610 and the diaphragm 620. The base plate 610 includes a first opening 615 that provides for communication between a first fluid passageway 613 and the fluid chamber 680. The base plate 610 includes a second opening 612 that provides for communication between a second fluid passageway 614 and the fluid chamber 680. In some examples, the diaphragm 620 can be actuated to move between a closed position in which the diaphragm 620 is proximate to the base plate 610 due to the deflection of the diaphragm 620, such that the volume of the chamber 680 is minimized, and an open position in which the base plate 610 is separated, or spaced apart from, the diaphragm 620 due to the deflection of the diaphragm 620, such that the volume of the chamber is maximized. When the diaphragm 620 is actuated to move from the closed position to the open position, fluid can be drawn into the chamber 680 through the first fluid passageway 613, and when the diaphragm 620 is actuated to move from the open position to the closed position, fluid can be expelled from the chamber 680 through the second fluid passageway 614. Repeatedly actuating the diaphragm between the closed and open position allows fluid to be pumped through the pump device 600, from the first fluid passageway 613 to the second fluid passageway 614 via the fluid chamber 680.

In some implementations, the pump device 600 can include one or more foil plates 650 and 652 to control the flow of fluid into and out of the pump device 600. The foil plates 650, 652 can include one-way check valves that operate to permit fluid to flow in one direction through the values but not in an opposite direction. The one-way check valves defined by the one or more foil plates can be positioned in, or in fluid connection with, a fluid passageway 613, 614 of the pump device 600. In some examples, a check valve is positioned in, or in fluid connection with, a portion of a fluid passageway 613, 614 so as to inhibit the unintended flow of fluid through the pump device in the event of a fluctuation, or spike in pressure. In some examples, a check valve is positioned in a fluid passageway 613, 614 so as to counteract a back pressure that would otherwise overcome the closing pressure and cause unintentional flow through the pump device 600. In some example implementations, a first check valve defined by one or more foil plates 650, 652 is positioned in, or in fluid connection with (e.g., at a first opening 611 of), a first fluid passageway 613 of the pump device and is configured to permit fluid to easily flow from the first fluid passageway 613 into the chamber 680 but to prevent or inhibit the flow of fluid from the chamber 680 into the passageway 613. In some example implementations, a second check valve defined by one or more foil plates 650, 652 is positioned in, or in fluid connection with (e.g., at a first opening 612 of), a second fluid passageway 614 of the pump device 600 and is configured to permit fluid to easily flow from the chamber 680 into the second fluid passageway 613 but to prevent or inhibit the flow of fluid from the passageway 613 into the chamber 680.

Application of an alternating current (AC) voltage to the piezoelectric element 640 can cause the diaphragm 620 of the pump device 600 to oscillate between a first position that defines the closed position of the chamber 680, in which the diaphragm 620 is proximate to the base plate 610 and the volume of the chamber 680 is minimized, and a second (e.g., domed) position that defines the open position of the chamber 680, in which the diaphragm 620 is separated from the base plate and the volume of the chamber 680 is maximized. As the diaphragm 620 of the pump device 600 oscillates between a first position and the second position, fluid is drawn into the chamber 680 from the first passageway 613 and is expelled from the chamber 680 into the second passageway 614. As the diaphragm 620 of the pump device 600 oscillates between a first position and the second position, the one-way check valves defined by the one or more foil plates 650, 652 prevent or inhibit fluid from flowing from the chamber 680 into the first passageway 613 and prevent or inhibit fluid from flowing into the chamber 680 from the second passageway 614. Thus, the application of the AC voltage to the piezoelectric element 640 causes the pump device 600 to pump fluid from the first passageway 613 to the second passageway 614.

The frequency of the AC voltage applied to the piezoelectric element 640 can determine an oscillation mode of the piezoelectric element 640. In some implementations, the frequency of the AC voltage is selected to excite a lowest-order mode in which the center of the circular piezoelectric element 640 experiences the greatest extent of movement during an oscillation cycle, such that an amount of fluid pumped during an oscillation cycle is maximized compared to other oscillation modes.

The piezoelectric element 640 can be controlled to cause fluid to be pumped by device 600, for example, by repeatedly changing a volume of the fluid chamber 680 by deforming the deformable diaphragm 620 to pump fluid from the fluid reservoir to the inflatable member.

The volume of the chamber 680 can be determined, at least in part, by the shape, geometry, and material properties of the components used to form the chamber 680, including, for example, the base plate 610 and the deformable diaphragm 620. In some cases, a relatively larger volume of the chamber 680, for an approximately constant diameter of the chamber, can result in more fluid being pumped in each open/close cycle of the pump device 600. To achieve a relatively larger volume of chamber 680, the deformable diaphragm can be deformed or biased into a non-flat dome-shaped configuration before it is attached to the piezoelectric element 640.

In some implementations, before the diaphragm 620 is placed in attached to the piezoelectric element 640, a voltage can be placed across the electrodes 690 attached to the piezoelectric element 640 to configure the piezoelectric element 640 in the domed configuration that is assumes when the fluid chamber is in the open position (See FIG. 4D). Then, the diaphragm can be placed in contact with the piezoelectric element while the piezoelectric element 440 is in its domed configuration, and the epoxy can be cured when the piezoelectric element and the diaphragm 420 are in the domed configuration, which can reduce stress on the adhesive bond between the diaphragm 420 and the piezoelectric element 440.

Referring again to FIG. 2, although considerable effort is expended to maintain the cleanliness of the components of the system and the purity of the fluid used within the system, it is still possible that some small amounts of foreign matter can contaminate the fluid within the system. For example, when the reservoir 202, the inflatable members 204, and the housing 210 are implanted and connected (e.g., by conduits 203, 207) within a patient, it is possible that some contamination enters the fluidic system. In addition, it is possible that, once implanted within a patient, that small amounts of material disintegrate from walls of the reservoir 202, inflatable member 204, housing 210 and conduits 203, 207 and become suspended within fluid that flows within the system 200. Because of the small internal dimensions of the pumps and valves used within the fluidic system, the existence of particles of foreign matter suspended within the fluid flowing within the system poses a risk of clogging or damaging one or more of the pumps and valves, which may lead to malfunction of the system 200. To mitigate the effect of any particulate matter suspended within the fluid that flows within the system 200, the fluidic path can include one or more filters that block, or reduce the amount of, particulate matter that enters the pumps and valves of the system. In some implementations, the filters can be included in a fluid pathway of a pump or valve.

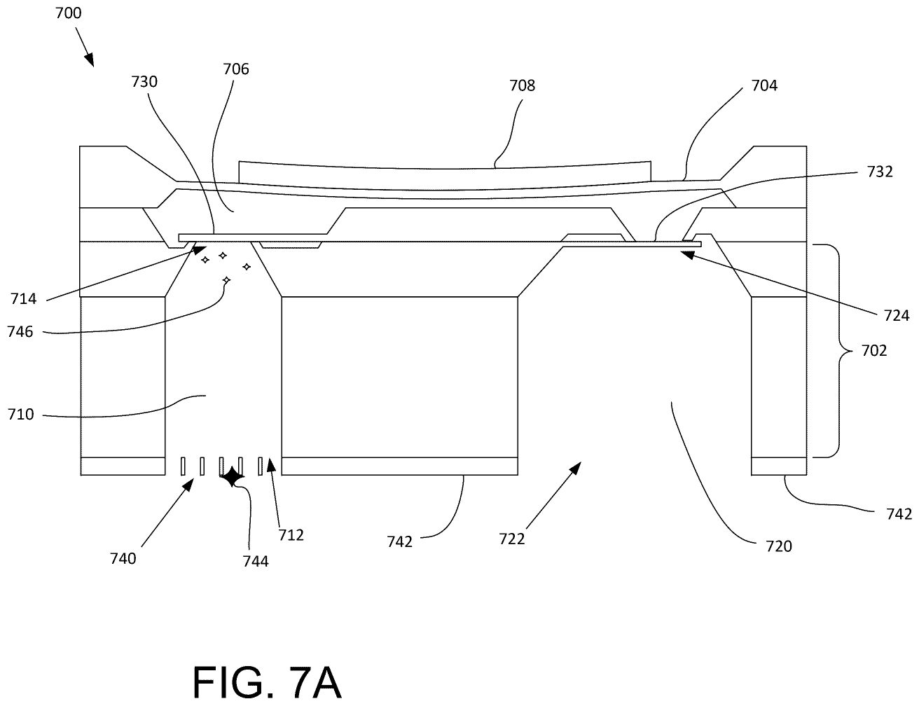

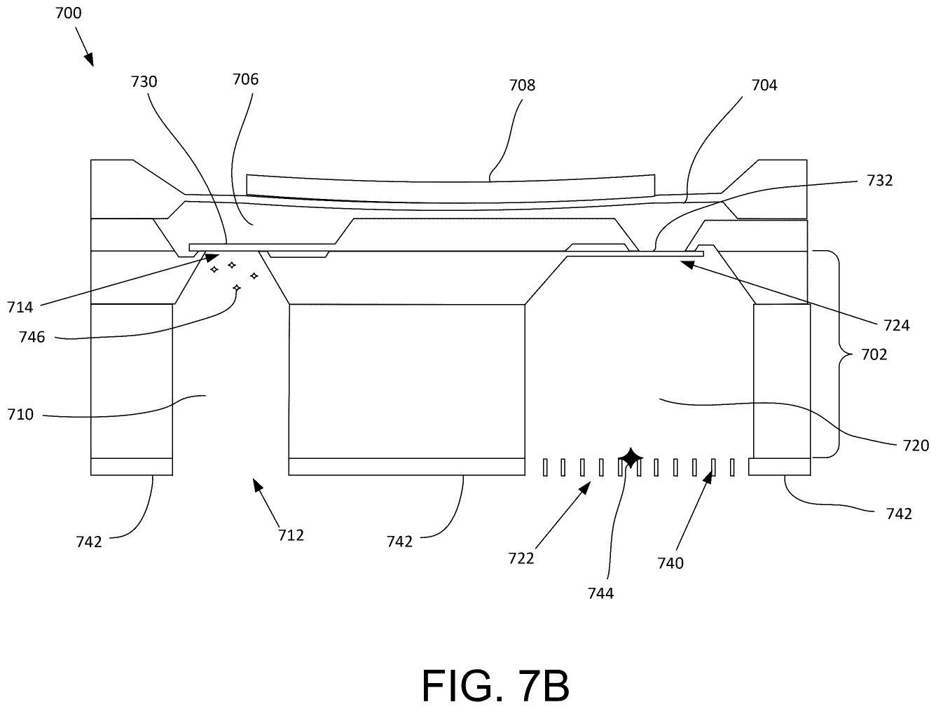

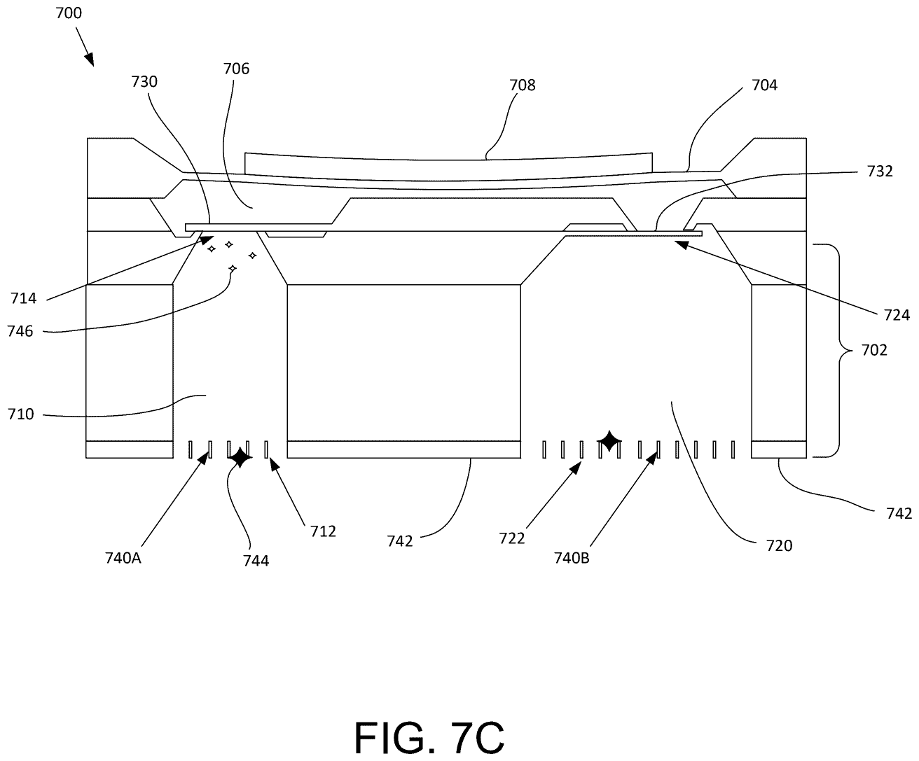

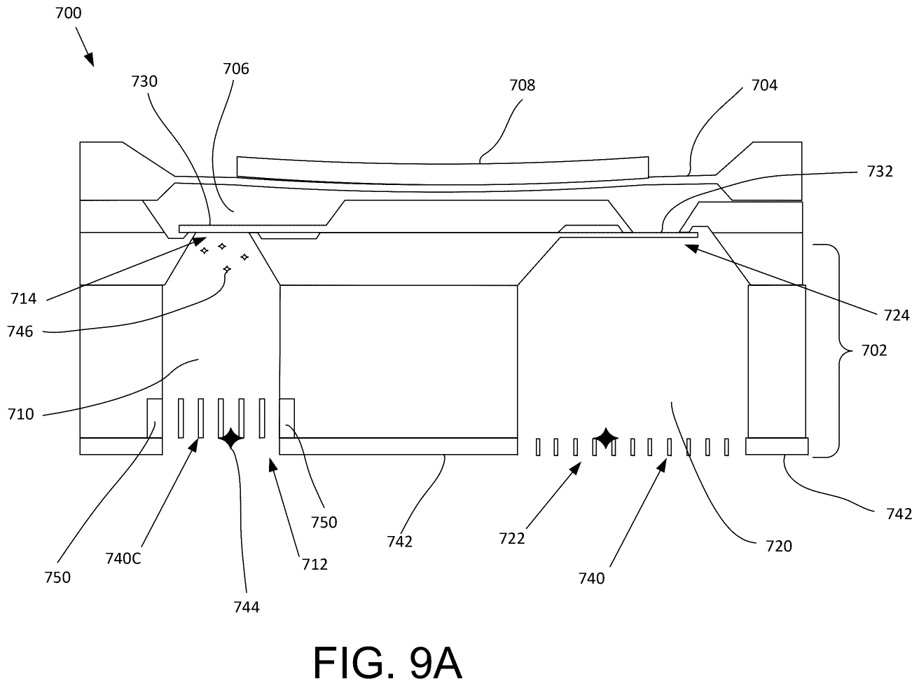

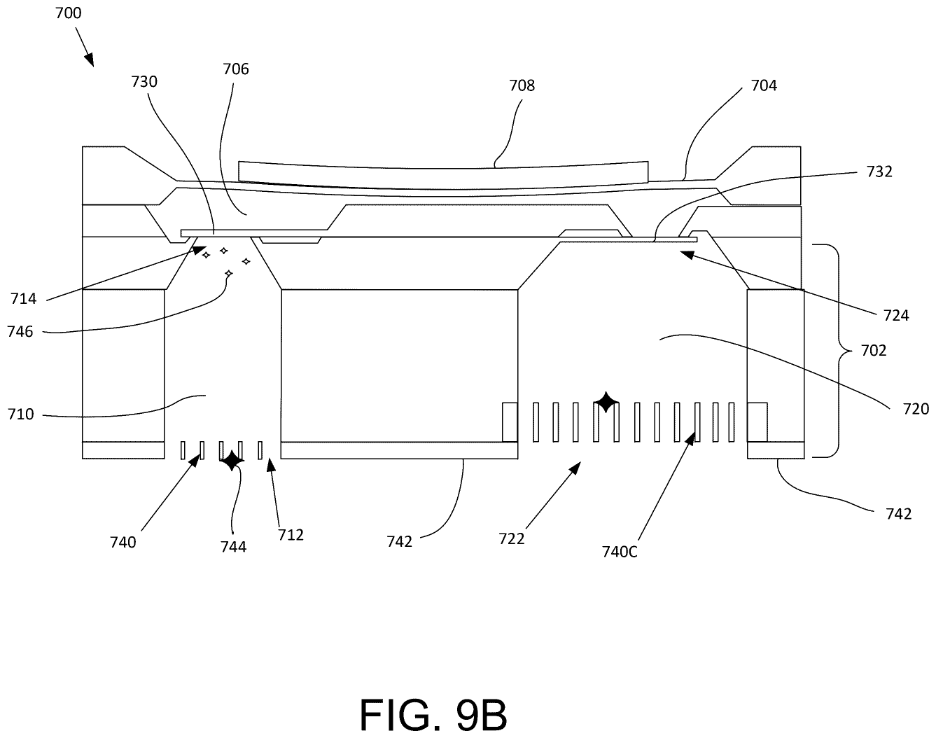

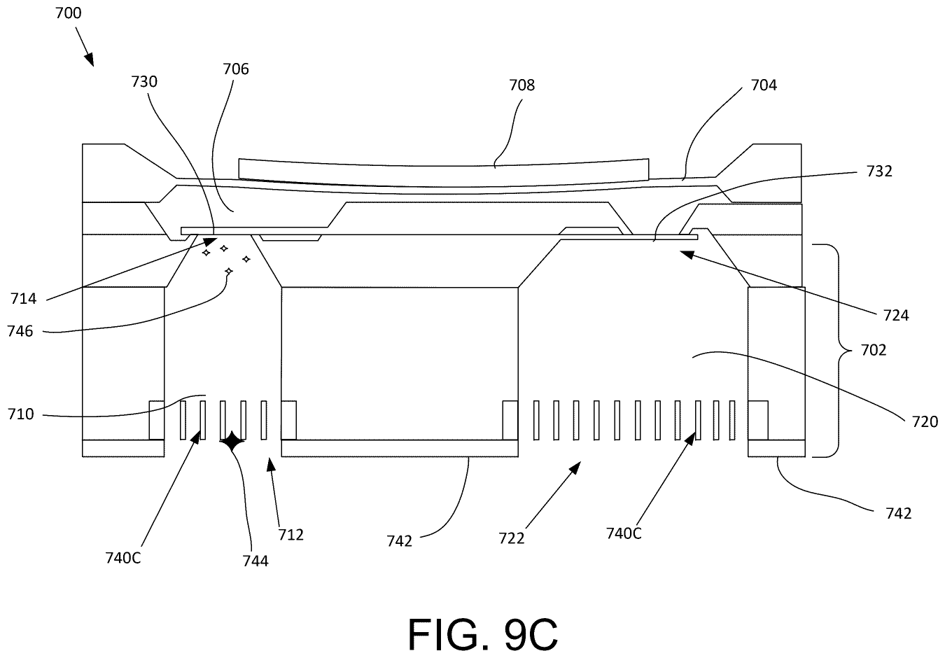

FIGS. 7A, 7B, 7C, 9A, 9B, and 9C are cross-sectional views of example pump devices 700 that includes a filter for capturing particulate matter in the fluid flow and/or for blocking the particulate matter from entering certain parts of the fluidic system (e.g., for blocking particulate matter from entering a pump chamber of the device). The example pump device 700 shown in FIGS. 7A, 7B, 7C, 9A, 9B, and 9C are examples of a fluid control device, or a fluidic component, included in the fluid control system 206 of the example electronically controlled fluid manifold 230 described above.

In the example arrangements shown in FIGS. 7A, 7B, 7C, 9A, 9B, and 9C, the example pump device 700 includes a base plate 702 defining a base portion of the pump device 700. A diaphragm 704 is positioned above the base plate 702, and a fluid chamber 706 is defined between the base plate 702 and the diaphragm 704. A piezoelectric element 708 is positioned on the diaphragm 704. The piezoelectric element can be electrically powered (e.g., by a battery of the implantable fluid-operated inflatable device) to drive the diaphragm 704 to pump fluid through the pump device 700. The diaphragm 704 can include a thin metal foil, whose shape can be repeatably deformed in response to movement by the piezoelectric element 708. In some implementations, the diaphragm 704 can include titanium material.

The base plate 702 can define a first fluid passageway 710 through which fluid can flow from a fluid reservoir into the fluid chamber 706. The first fluid passageway 710 can include an opening 712 at a first end of the passageway 710, which is distal to the fluid chamber 706, and can include an opening 714 and a second end of the passageway 710, which is proximate to the fluid chamber 706. The base plate 702 can define a second fluid passageway 720 through which fluid can flow from the fluid chamber 706 to an inflatable member. The second fluid passageway 720 can include an opening 722 at a first end of the passageway 720, which is distal to the fluid chamber 706, and can include an opening 724 and a second end of the passageway 720, which is proximate to the fluid chamber 706. In some implementations, the first fluid passageway 710 and the second fluid passageway 720 can be tapered, such the passageways 710, 720 have larger cross-sectional areas at the ends 712, 722 of the passageways that are distal to the fluid chamber 706 than at ends of the passageways that are proximate to the fluid chamber.

The pump device 700 can include a first flexible flap 730 that includes a portion that has an area that is greater than an area of the passageway opening 714 that is proximate to the fluid chamber 706 and that covers the opening, such that the first flexible flap 730 is configured to seal against portions of the base plate that defines the opening 714 of the first fluid passageway 710 to close the opening 714 when a fluid pressure in the fluid chamber 706 is greater than a fluid pressure of fluid in the first fluid passageway 710. The flexible flap 730 can be secured to the base plate over a portion of its extent but can have a portion that is unsecured, such that at least a portion of the flexible flap is configured to be pushed away from one or more walls of the fluid passageway 710 that defines the opening 714 when a fluid pressure of fluid in the first fluid passageway 710 is greater than a fluid pressure in the fluid chamber 706. In this manner, the flexible flap 730 operates to allow fluid to flow from the first fluid passageway 710 into the fluid chamber 706 but to block the flow of fluid from the fluid chamber 706 into the first fluid passageway 710. The flexible flap 730 can be made of a variety of materials including, for example, titanium, elastomeric material, plastic material, etc.

The pump device 700 can include a second flexible flap 732 that includes a portion that has an area that is greater than an area of the passageway opening 724 that is proximate to the fluid chamber 706 and that covers the opening, such that the second flexible flap 732 is configured to seal against portions of the base plate that defines the opening 724 of the second fluid passageway 720 to close the opening 724 when a fluid pressure in the fluid chamber 706 is greater than a fluid pressure of fluid in the second fluid passageway 720. The flexible flap 732 can be secured to the base plate over a portion of its extent but can have a portion that is unsecured, such that at least a portion of the flexible flap is configured to be pushed away from one or more walls of the second fluid passageway 720 that defines the opening 724 when a fluid pressure in the fluid chamber 706 is greater than a fluid pressure of fluid in the second fluid passageway 720. In this manner, the flexible flap 732 operates to allow fluid to flow from the fluid chamber 706 into the second fluid passageway 720 but to block the flow of fluid from the second fluid passageway 720 into the fluid chamber 706. The flexible flap 732 can be made of a variety of materials including, for example, titanium, elastomeric material, plastic material, etc.

With the flexible flaps 730, 732 configured in this way to allow fluid to flow in a first direction from the first fluid passageway 710 into the fluid chamber 706 and out of the fluid chamber into the second fluid passageway 720 but not in a direction opposite to the first direction, repeated expansion and contraction of the volume of the fluid chamber 706 in response to the piezoelectric element 708 operating on the deformable diaphragm 704 can cause fluid to be pumped from a reservoir fluidically connected to the first fluid passageway 710 to an inflatable member that is fluidically connected to the second fluid passageway 720.

The pump device 700 can include a fluid filter 740 that is located within, or at the end 712 of, the first fluid passageway 710 or that is located within, or at the end 722 of, the second fluid passageway 720. The fluid filter 740 can operate to block, for example, debris, foreign matter, particulates suspended in the fluid flowing through the device 700 from passing through the first fluid passageway 710 and into the fluid chamber 706 and/or from exiting the second fluid passageway 720. For example, as shown in FIG. 7A, a fluid filter 740 is located at the opening 712 into the first fluid passageway 710. As shown in FIG. 7B, a fluid filter 740 is located at the opening 722 into the second fluid passageway 720. As shown in FIG. 7C, a fluid filter 740A is located at the opening 712 into the first fluid passageway 710, and a fluid filter 740B is located at the opening 722 into the second fluid passageway 720.

In some implementations, the fluid filter 740, 740A, 740B can include a metal foil (e.g., a titanium foil, having a pattern of openings that permit fluid to flow through the openings but that block particulates having a characteristic size larger than a threshold size from flowing through the opening. For example, particulates 744 having a characteristic size (e.g., minimum transverse extent) that is greater than a threshold size defined by the size (e.g., diameter) of the openings can be blocked by the filter 740, while particulates 746 and a characteristic size smaller than the threshold size can pass through the filter 740.

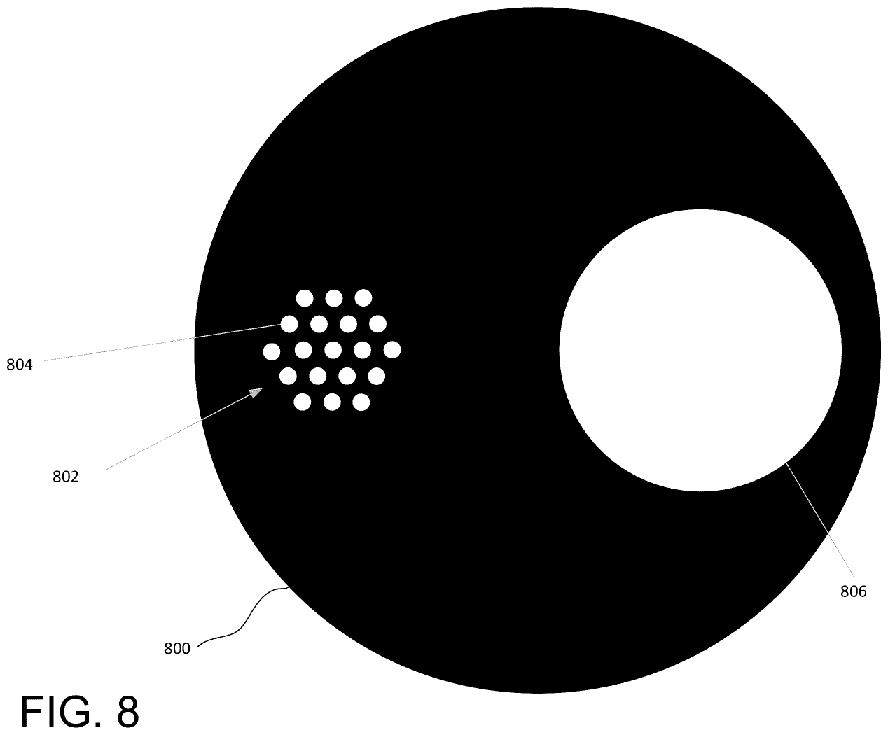

FIG. 8 is a schematic end view of a filter foil 800. In some implementations, the filter foil 800 can be made of metal (e.g., titanium) and can have a first section 802 that includes a plurality of openings 804. The openings can have a variety of different shapes, including circular, oblong, square, rectangular, hexagonal, etc. The plurality of openings 804 can be arranged in a regular or irregular pattern. For example, the openings 804 can be arranged in a two-dimensional hexagonal pattern, as shown in FIG. 8, or in a square pattern, or another type of regular or irregular pattern.

The plurality of openings 804 can be formed in the filter foil 800 in a number of different ways. For example, in some implementations, the pattern of openings can be mechanically stamped into the metal foil 800. In some implementations, the pattern of openings 804 can be laser etched into the metal foil 800. In some implementations, the pattern of openings can be chemically etched (e.g., through a lithographic process) into the metal foil 800.

Referring again to FIG. 7A and also to FIG. 8, the section 802 that includes the plurality of openings 804 can be arranged on the filter foil 800 so that the pattern of openings 804 is aligned with the opening 712 of the first fluid passageway 710 when the filter foil 800 is attached to the base plate 702. The filter foil 800 also can include an opening 806 in the filter foil that is aligned with the opening 722 of the second fluid passageway 720 of the base plate 702 when the filter foil is attached to the base plate.

In some implementations, the filter foil 800 can be welded to the base plate 702. For example, when the base plate includes titanium and the filter foil 800 includes titanium, the filter foil 800 can be welded to the titanium base plate 702. Prior to attempting (e.g., welding) the filter foil 800 to the base plate 702, the filter foil 800 can be positioned relative to the openings 712, 720 in the base plate, such that the first section 802 of the filter foil, which includes the plurality of openings 804, is positioned at the end of the first fluid passageway 710 and such that the opening 806 in the filter foil 800 is positioned at the end of the second fluid passageway 720. Similarly, when a filter foil is attached to the base plate shown in FIG. 7B, a section of the filter foil having a plurality of openings can be aligned with the end of the second fluid passageway 720, and a larger opening in the filter foil 800 in the aligned with the end of the first fluid passageway 710. Similarly, when a filter foil is attached to the base plate shown in FIG. 7C, a first section having a plurality of openings can be aligned with the end of the second fluid passageway 720 and a second section having a plurality of openings can be aligned with the end of the first fluid passageway 710.

In implementations in which the first fluid passageway 710 and the second fluid passageway 720 are tapered, such the passageways 710, 720 have larger cross-sectional areas at the ends 712, 722 of the passageways that are distal to the fluid chamber 706 than at ends of the passageways that are proximate to the fluid chamber, filters 740, 740A, 740B positioned at the distal ends of the fluid passageways 710, 720 can have cross-sectional areas that are greater than the cross-sectional areas of the openings 714, 724 between the passageways 710, 720 and the fluid chamber 706. Because of this the area of the filter that is active for trapping particulate matter can be larger than the areas of the openings 714, 724 between the passageways 710, 720 and the fluid chamber 706. In some implementations the flow of fluid through the filter 740, 740A, 740B can be reversed to dislodge some of the particulate matter that has been trapped by the filters from the filters.

For example, referring again to FIG. 3A, a fluid conduit C1 can be provided between a downstream side of valve V2 and a pump P1. When the pump P1 is configured similarly to the pump shown in FIG. 7A, with a filter 740 at the end of the fluid passageway 710, the fluid conduit C1 can be connected to the first fluid passageway 710, so that when fluid is pumped from inflatable member(s) 204 to the reservoir 202 some of that fluid is pumped into the fluid passageway 710 of pump P1. Then, with valve V1 closed, the fluid that enters the first fluid passageway 710 of pump P1 can flow out of the distal end 712 of the first fluid passageway 710 and back to the reservoir 202. The fluid that flows out of the distal end 712 of the first fluid passageway 710 can flush debris and particulate matter out of the filter 740. In some implementations, the conduit C1 can include a one-way valve that allow fluid to pass from valve V2 to pump P1 but not in the opposite direction. Other such fluid connections, for example, conduit C2 of FIG. 3A, can be used to flush debris and particulate matter out of filters used in the fluid control system.

The example pump devices 700 shown in FIGS. 7A, 7B, 7C include filters 740, 740C for blocking particulate matter in the fluid from entering a pump chamber of the device or for circulating in the fluidic system in which the pump devices operate. The filter 740 shown in FIG. 7A is disposed at the distal end 712 of the first fluid passageway 710, and the filter 740 shown in FIG. 7B is disposed at the distal end 722 of the second fluid passageway 720. These filters 740 can be similar to the filters 740, 740B, 740B shown in FIGS. 7A-15C, in that the filters 740 can include a plurality of openings in a foil, where the size of the openings is selected to block the passage of particles having a characteristic size greater than a threshold size and to allow fluid and particles having a characteristic size less than the threshold size to pass through the openings.

In some implementations, the example pump devices 700 shown in FIGS. 7A, 7B, 7C can include filters 740C disposed within the first fluid passageway 710 or within the second fluid passageway 720, for example, between the first end 712 of the first fluid passageway 710 and the opening 714 at the second end of the first fluid passageway 710 and/or between the first end 722 of the second fluid passageway 720 and the opening 724 at the second end of the second fluid passageway 720. For example, as shown in FIG. 7A, the example pump device 700 can include a filter 740C disposed within the first fluid passageway 710. In another example, as shown in FIG. 7B, the example pump device 700 can include a filter 740C disposed within the second fluid passageway 720. In another example, as shown in FIG. 7C, the example pump device 700 can include a filter 740C disposed within the first fluid passageway 710 and another filter 740C disposed within the second fluid passageway 720.

Referring to FIG. 7A, the filter 740C can include an outer frame 750 that supports material within the frame that includes a plurality of small openings or passages through which fluid can pass but which have a threshold size that blocks particles having a characteristic size greater than the threshold size from passing through the filter 740C.

The outer frame 750 can be secured to the base plate 702 that defines the first fluid passageway 710. In some implementations, the base plate 702 can define a receptacle that receives the outer frame 750. In some implementations, the receptacle can have a lateral extent (e.g., a diameter) that is greater than the lateral extent of the first fluid passageway 710, such that when the outer frame 750 is disposed in the receptacle, an inner wall of the outer frame has a lateral extent that is similar to the lateral extent of the first fluid passageway 710. In some implementations, the outer frame can be press fit into the receptacle. In some implementations the outer frame 750 can be welded to the portion of the base plate 702 that defines the receptacle. In some implementations, after the outer frame 750 of the filter 740C is placed in the receptacle, a foil 742 can be placed over the outer frame 750 and then attached (e.g., welded) to the base plate 702.

In different implementations, the outer frame 750 can be made of different materials. For example, if the outer frame 750 is to be welded to a titanium base plate 702, the outer frame 750 can be made of titanium. In another example, if the outer frame 750 is to be securely press fit into a receptacle, the outer frame 750 can be made of a compliant material, for example, plastic, rubber, etc.

The material of the filter 740C supported by the outer frame 750, which includes a plurality of small openings or passages through fluid passes, can be made of different materials, which need not be identical or similar to the materials of the outer frame 750. For example, the material can include metal (e.g., titanium, gold, etc.). In another example the material can include ceramic material. In another example, the material can include plastic.

In some implementations, the thickness of the material of the filter, which includes the plurality of small openings or passages through which fluid passes, in the direction of the fluid flow through the filter can be greater than three times the mean lateral extent of the openings or passages through which the fluid passes. Thus, the openings or passages of the materials can operate more as tubes through which the fluid passes than as apertures in a thin plane of material. In some implementations, walls of the openings or passages of the material can be textured or treated to promote the adhesion of particulate matter, while also permitting the fluid to pass through the openings or passages. For example, the walls of the openings or passages can have a surface texture or roughness that facilitates the adhesion of particulate matter, and the service of the openings or passages can include a hydrophobic coating to encourage the passage of fluid through the openings or passages.

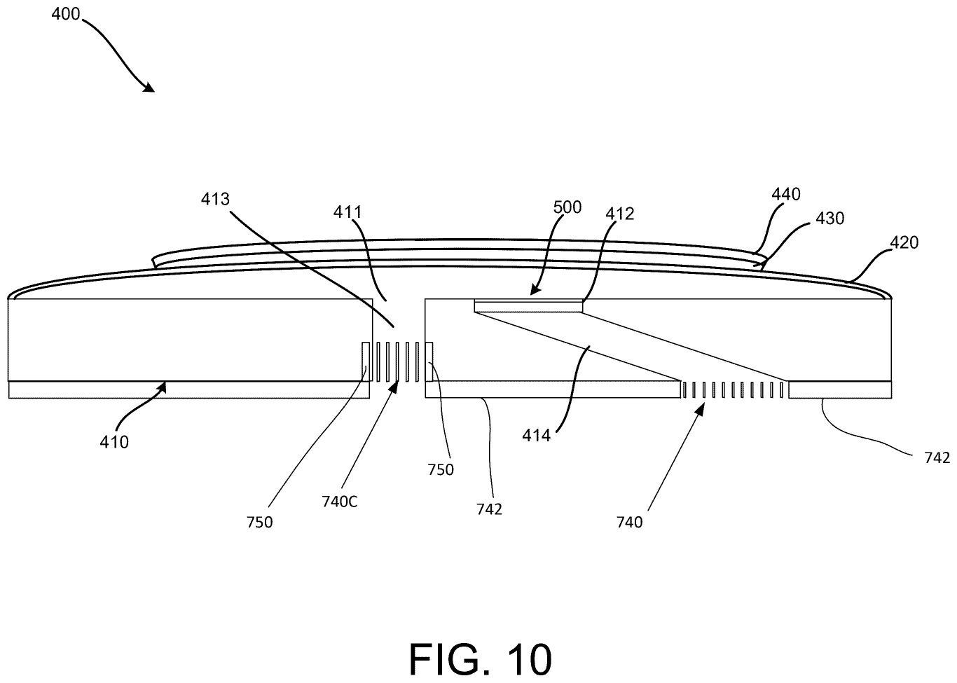

In addition to being used in the pumps described herein, the filters described herein also can be used in the valves described herein. For example, FIG. 10 is cross-sectional view of the valve device 400 shown in FIG. 5A, but also including a filter 740 located at an end of the second fluid passageway 414 and a filter 740C located within the first fluid passageway 413. The filters described herein also may be utilized in other valve structures described herein.

It is desirable that the implantable fluid-operated inflatable device described herein can be implanted in a patient and used to provide safe, reliable, and successful therapeutic treatment to the patient for many years, for example, 10 or more years. However, a challenge with meeting this reliability goal is that the piezoelectric elements used in combination with the thin metal diaphragms to provide the pumps and valves of the fluid-operated inflatable device, as described herein, are susceptible to a number of processes and risks that can lead to degradation and/or failure of the piezoelectric elements and the pumps and valves with which they are associated.

For example, as the piezoelectric elements are used over many cycles during the lifetime of the implantable device 100, 200, the crystal structure of the piezoelectric elements experiences mechanical stress as the dimensions and shape of the piezoelectric elements changes in response to the application of different voltages to the piezoelectric elements. The mechanical stress on the material of a piezoelectric element may cause cracks to form in the material, which may affect the electrical properties of the material. For example, a crack in the material may cause a microscopic change in the resistivity of the material at the location of the crack. A small crack in the material of the piezoelectric element may cause the element to move or change its shape by a slightly smaller amount for the same change in voltage applied to the piezoelectric element than before the crack existed. This may not cause the piezoelectric-operated pump to fail but may cause the piezoelectric-operated pump to pump a slightly smaller amount of fluid during each pumping cycle. However, as the size of the crack grows or as the number of cracks in the material proliferate over the lifetime of the device, the electromechanical properties of the piezoelectric element may change enough, so that the piezoelectric element may no longer function as intended in the implantable device.

In another example, referring again to FIG. 4D, if the bond between the diaphragm 420 and the base plate 410 is not completely fluid tight, then liquid from within the chamber between the diaphragm and the base plate may leak from the fluid chamber side the diagram to the side of the diaphragm that is attached to the piezoelectric element 440, and the liquid may cause a short circuit in the piezoelectric element. The short circuit may cause the performance of the piezoelectric-operated pump to degrade (e.g., for the pump to pump a smaller amount of fluid during each pumping cycle) or may cause the pump to fail completely.

In another example, even if the chemical properties of the piezoelectric element remain unchanged, a buildup of foreign material in the small passageways, chambers, and conduits of the implanted device may clog one or more elements of the device (e.g., a valve, a filter, a pump) and impede the flow of fluid within the device to a degree that the piezoelectric-operated device is not designed to handle. This also may cause the performance of the piezoelectric-operated pump to degrade (e.g., for the pump to pump a smaller amount of fluid during each pumping cycle) or may cause the pump to fail completely.

In the face of such challenges to, and failure modes of, piezoelectric elements that are used to operate the pumps and valves of the implantable fluid-operated inflatable device 100, 200, techniques are disclosed herein for monitoring the performance and health of the piezoelectric elements and for automatically implementing corrective action to preserve the effectiveness of the pumps and valves and/or the safety of the patient in whom the inflatable devices implanted when significant changes in the performance or health of a piezoelectric element is detected.

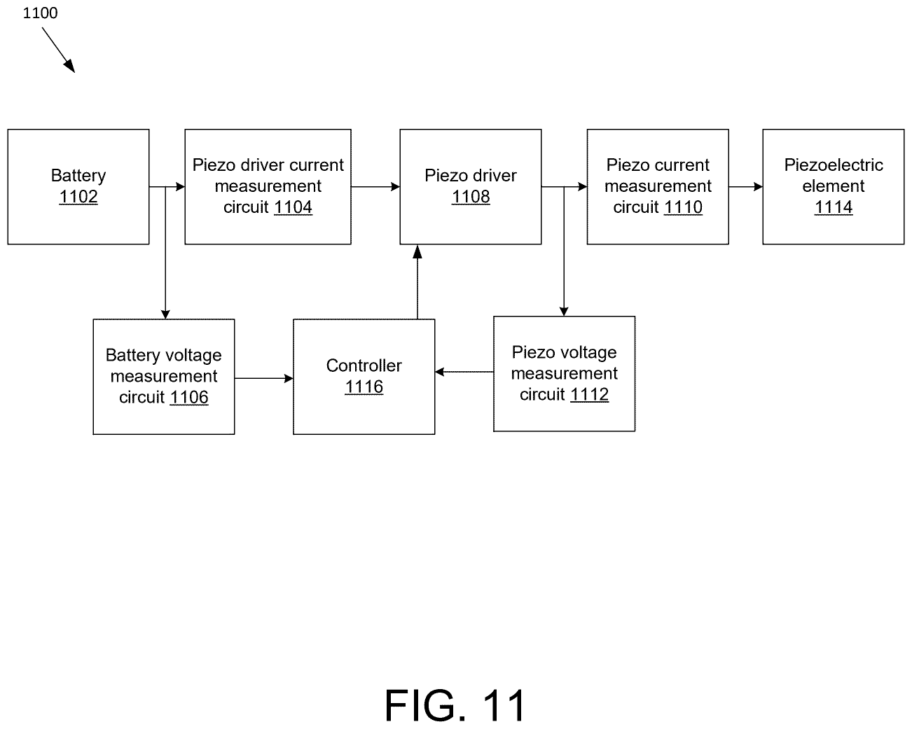

FIG. 11 is a schematic block diagram of a system 1100 for driving a piezoelectric element 1114 of a piezoelectric-operated pump or valve and for monitoring and controlling the performance of the piezoelectric element. The system 1100 includes a battery 1102 that is configured to store electrical energy that can be used to drive the piezoelectric element 1114. A piezoelectric driver 1108 is electrically connected to the battery 1102 and to the piezoelectric element 1114. The piezoelectric driver 1108 includes electronic circuitry (e.g., analog and/or digital electronic circuitry) that is configured for receiving electrical energy from the battery 1102 and for generating a waveform of electrical energy that is provided to the piezoelectric element to drive the piezoelectric element.

In some implementations, the battery 1102 can provide electrical energy at a maximum voltage of 5 V or less, for example, at a maximum of 4.4 V or less to the piezoelectric driver 1108. The driver 1108 can step up the voltage and can output a waveform having a peak-to-peak voltage of greater than 50 V, for example, 100 V, to the piezoelectric element 1114. In some implementations, the driver 1108 can include step up transformer circuitry configured for receiving a first voltage signal from the battery 1102 and for outputting a second voltage signal to the piezoelectric element, where the second voltage is greater than the first voltage.

When the piezoelectric element 1114 is associated with a pump of the implantable inflatable device 100, 200, the driver 1108 can output a periodic waveform that is used to repeatedly change a volume of a fluid chamber to cause fluid to be pumped through the fluid chamber from one location to another, for example, from a reservoir to an inflatable member or from the inflatable member to the reservoir. In some implementations, a frequency of the periodic waveform can be between 30 Hz and 60 Hz, for example, 40-50 Hz. In some implementations, the periodic waveform can be a sine wave. In some implementations, the periodic waveform can include a series of square pulses. In some implementations, the periodic waveform can include a repeated series of waves provided to the piezoelectric element 1114, where the waves have a voltage that varies over time according to a function V=V(t) and where, unlike a sine wave, the second derivative of V divided by V (i.e., V″(t)/V(t)) is not equal to one but where, unlike a square wave, V(t) does not include discontinuities, at which the first derivative of V(t) approaches infinity. When comparing two waveforms having an identical frequency and an identical peak-to-peak amplitude, a first waveform in the form of a sine wave may be more energy-efficient, in terms of preserving energy in the battery 1102, for driving the piezoelectric element 1114 than a second waveform in the form of a series of square pulses. More generally, a first waveform V₁(t) may be more energy-efficient, in terms of draining energy from the battery 1102, for driving the piezoelectric element 1114 to pump a certain volume of fluid than a second waveform V₂(t) when the maximum of V″₁(t)/V₁(t) is less than the maximum of V″₂(t)/V₂(t).

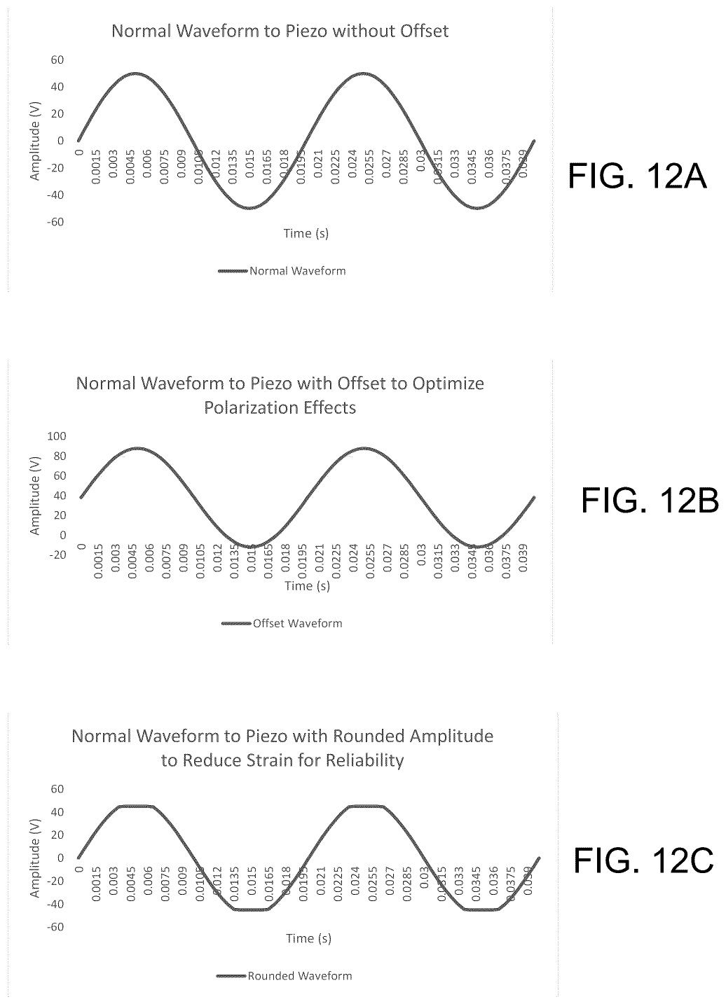

FIG. 12A is a graph of the voltage amplitude of an example waveform that can be a provided by the driver 1108 to the piezoelectric element 1114 to drive the piezoelectric element to cause a pump associated with the piezoelectric element to pump fluid. The waveform has an amplitude that varies over time according to a function V(t) that is approximated by a sine wave. In the example waveform of FIG. 12A, the voltage varies from −50 V to +50 V and has a frequency of 50 Hz.

FIG. 12B is a graph of the voltage amplitude of another example waveform that can be a provided by the driver 1108 to the piezoelectric element 1114 to drive the piezoelectric element to cause a pump associated with the piezoelectric element to pump fluid. The waveform has an amplitude that varies over time according to a function V(t) that is approximated by a sine wave having a frequency of 50 Hz. In contrast to the example waveform of FIG. 12A, in the example waveform of FIG. 12B, the average voltage over time is offset from zero, and the voltage varies from −12 V to +88 V. By offsetting the average voltage from zero, the poling of the piezo can be properly maintained.

FIG. 12C is a graph of the voltage amplitude of another example waveform that can be a provided by the driver 1108 to the piezoelectric element 1114 to drive the piezoelectric element to cause a pump associated with the piezoelectric element to pump fluid. The waveform has an amplitude that varies over time according to a function V(t), has a frequency of 50 Hz, and a voltage that varies from −45 V to +45 V. In contrast to the example waveform of FIG. 12A, the example waveform of FIG. 12C is not approximated by sine wave but rather is approximated by a sine wave having a time-averaged value of zero, with the peak-to-peak amplitude of 100 V, except that for the times at which the amplitude would be greater than +45 V the amplitude is held fixed at a plateau of +45 V and except that for the times at which the amplitude would be less than −45 V the amplitude is held fixed at a plateau of −45 V. By including the +45 V and −45 V plateaus in the waveform, the waveform of FIG. 12C may be able to pump a substantially similar, or even a greater, amount of fluid as the waveform of FIG. 12A, while causing less mechanical strain on the material of the piezoelectric element 1114, which may increase the reliability and longevity of the piezoelectric element. Because the fluid that is pumped by the piezoelectric-operated pump has a nonzero viscosity, the slightly smaller range of motion induced in the piezoelectric element 1114 by the application of the waveform of FIG. 12C, as compared to the application of the waveform of FIG. 12A, may result in a negligible difference in the amount of fluid pumped per cycle when the waveform of FIG. 12C is used instead of the waveform of FIG. 12A. Therefore, including short, fixed-voltage plateaus at the extrema of the voltage values of the waveform may increase the reliability and longevity of the piezoelectric element, while maintaining the pumping efficiency of the piezoelectric-operated pump.

Referring again to FIG. 11, the system 1100 can include one or more monitor circuits configured for determining electrical parameters of the waveform that is provided by the driver 1108 to the piezoelectric element. For example, a current measurement circuit 1110 can measure an electric current drawn by the piezoelectric element 1114, and a voltage measurement circuit 1112 can measure a voltage of the waveform provided to the piezoelectric element 1114, while the piezoelectric element operates to pump fluid in the implantable device. In addition, the system 1100 can include one or more monitor circuits configured for determining electrical parameters of electrical energy provided from the battery 1102 to the driver 1108. For example, a battery voltage measurement circuit 1106 can output a measured voltage of the battery 1102, and a driver current measurement circuit 1104 can measure a current drawn from the battery 1102 by the driver 1108 while the driver drives the piezoelectric element 1114. A controller 1116 can receive signals indicating the parameters measured by the monitor circuits 1104, 1106, 1110, 1112 and can process the signals to diagnose the performance and status of the battery 1102 and the piezoelectric element 1114 to detect existing and/or impending faults or failures in the battery or piezoelectric element. When a fault or failure is detected, the controller 1116 may take corrective action, for example, by signaling the driver 1108 to change the waveform provided to the piezoelectric element to mitigate the effects of the fault or failure or by causing the communication module 108C to signal the external controller 120 to alert the patient, a physician, or a technician, about the existing or impending fault or failure.

In some implementations, the battery voltage measurement circuit 1106 and the driver current measurement circuit 1104 can be used, respectively, to measure the voltage provided by the battery 1102 and the current provided by the battery while the piezoelectric-operated pump is used to pump fluid into an inflatable member of the implantable device. After the battery 1102 has been fully charged, the measurements can be obtained and stored each time the inflatable member is inflated to its designed pressure.

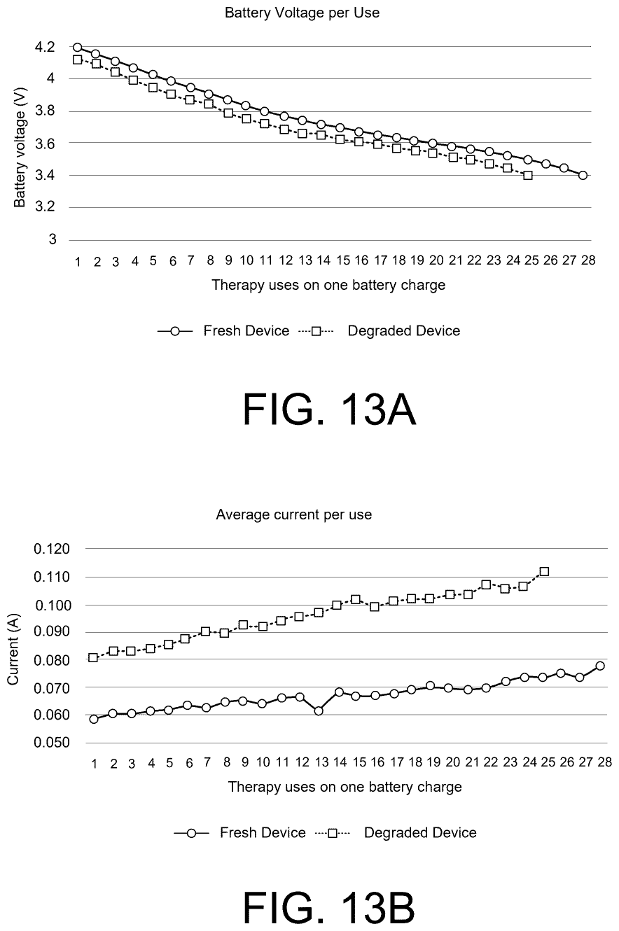

FIG. 13A is a graph of the measured battery voltage for each therapeutic inflation of the inflatable member that occurs after the battery 1102 has been fully charged. FIG. 13B is a graph of the measured current drawn from the battery 1102 during each therapeutic inflation of the inflatable member that occurs after the battery 1102 has been fully charged. The solid lines with the circular data points represent measured voltages for a new battery, and the dotted lines with the square data points represent measured voltages for a battery whose performance is degraded over time due to use. Once the battery reaches a minimum threshold value of, for example, 3.4 V the battery may not be able to supply sufficient power to carry out an additional inflation of the inflatable member. As shown in the example graphs of FIGS. 13A and 13B, a fresh battery may provide sufficient energy to carry out 28 inflations of the inflatable member on one battery charge, while the degraded battery may provide sufficient energy to carry out only 25 inflations of the inflatable member. As shown in FIG. 13A, the voltage of the fresh battery is higher when the battery is fully charged than the voltage of the degraded battery. For example, the voltage of the fresh battery is 4.2 V, and the voltage of the degraded battery is 4.1 V when the first inflation on a battery charge is carried out. Similarly, as shown in FIG. 13B, the current drawn by the driver 1108 when the battery is fresh is lower than when the battery is degraded. Thus, the measurement of the voltage of the battery, or the current drawn by the driver 1108 when the inflatable member is inflated by the piezoelectric-operated pump, when the battery is fully charged can be used by the controller 1116 to determine a relative degradation of the battery, so that the performance of the implantable device (e.g., the number of times the inflatable member can be inflated when the battery is fully charged) can be inferred.

Referring again to FIG. 13A, the slopes of the lines for the fresh battery and the degraded battery are substantially similar, which may indicate that the electrical properties of the piezoelectric element 1114 were substantially similar when the measurements represented by the circular data points and by the square data points were collected. However, if the electrical properties of the piezoelectric element 1114 change over time, the slope of a line representing battery voltage versus the number of therapy uses on one battery charge may change. For example, if the impedance of the piezoelectric element 1114 decreases due to microscopic cracks in the material of the piezoelectric element, then the amount of current needed to fully inflate the inflatable member may increase, and this would be manifested by a steeper negative slope of a line plotted in FIG. 13A. Thus, the measurement of the slope of the line plotted in a graph similar to that of FIG. 13A can be used by the controller 1116 to determine a relative degradation of the piezoelectric element 1114.

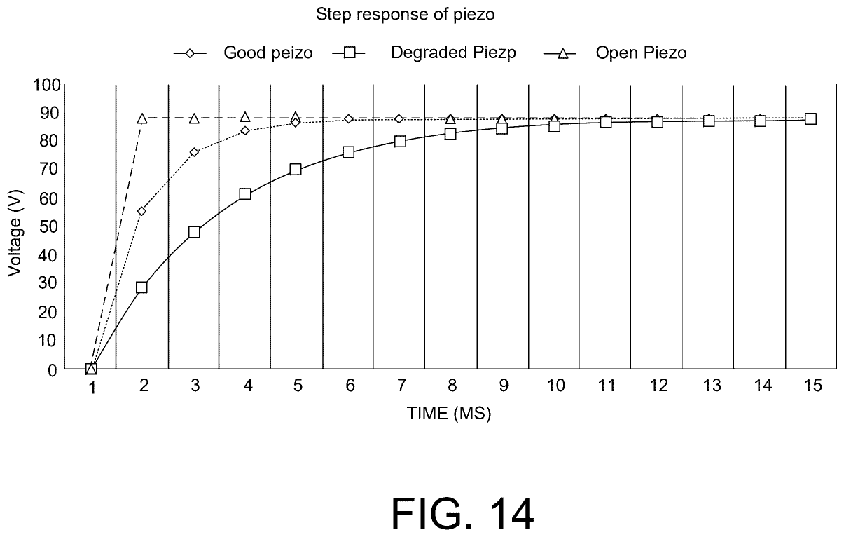

Other measurements from the monitor circuits 1104, 1106, 1110, 1112 also can be used to determine the status of the piezoelectric element 1114. For example, FIG. 14 is an example graph of a voltage on piezoelectric element 1114 as a function of time after a square wave pulse having an amplitude of 90 V is provided to the piezoelectric element. The piezoelectric element can be modeled as a capacitor that is charged as a function of time according to an equation, Q=CV_{b}[1−e^{-tlRC}], where Q is the charge on the capacitor, C is the capacitance of the capacitor, V_{b} is the voltage of the square wave pulse, t is the time after the pulse is applied, and R is the impedance of the capacitor. In FIG. 14, the voltage for a normal piezoelectric element 1114 is shown by the dotted line with diamond data points, and the voltage for a piezoelectric element that has been degraded by microscopic material cracks that lower the impedance of the element is shown by the solid line with square data points. The dashed line with triangle data points shows the voltage for a piezoelectric element that has experienced a fault causing the piezoelectric element to act as an open circuit. Thus, the driver 1108 can provide a test square pulse to the piezoelectric element, and the voltage response of the piezoelectric element can be measured by the voltage measurement circuit 1112. The measured response can be provided to the controller 1116, which can compare the measured response to a predetermined reference response to determine the current impedance of the piezoelectric element.

In another implementation, degradation of the electrical properties of the piezoelectric element 1114 can be determined by measuring the frequency spectrum of the complex impedance of the piezoelectric element. In some implementations, the driver 1108 can apply a test signal to the piezoelectric element 1114, in which a periodic waveform is applied to the piezoelectric element over a range of different frequencies, and the complex impedance of the piezoelectric element can be measured by the piezo voltage measurement circuit 1112 at the different frequencies.

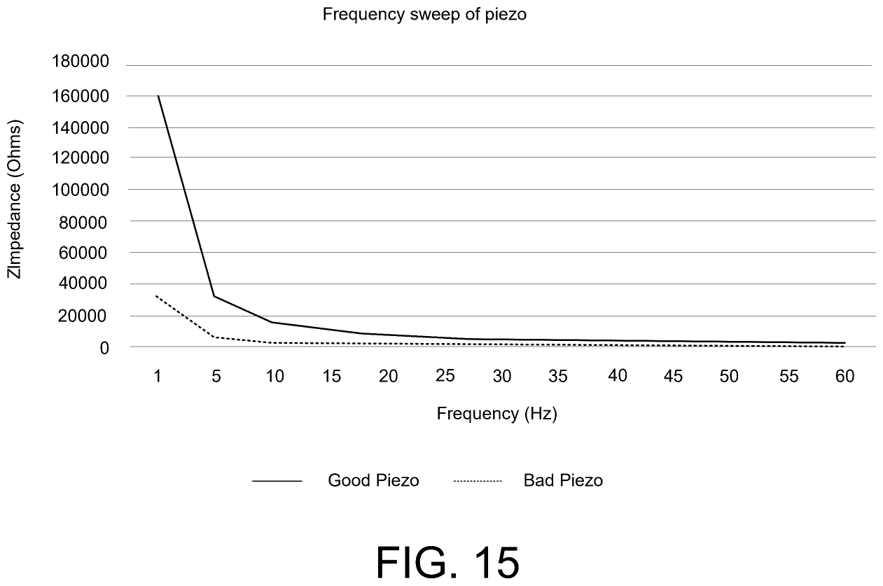

FIG. 15 is an example graph of the complex impedance of the piezoelectric element 1114 as a function of frequency over a range of frequencies from 0 to 60 Hz. In other implementations, the complex impedance can be measured over a wider range of frequencies, for example, from 0 to 20 kHz. As shown in FIG. 15, the complex impedance of a normal piezoelectric element as a function of frequency, as shown by the solid line, is quite different from the complex impedance, as a function of frequency, of a piezoelectric element that has been degraded by microscopic cracks, and resulting electrical shorts, in the material of the piezoelectric element.

When the battery 1102 and the piezoelectric element 1114 are not degraded and operate normally, the performance of the implantable device nevertheless can suffer due to a change in the flowrate of fluid within the implantable system, which may result from restrictions on the fluid flow due to debris in the system that may clog fluidic passageways of the implantable device. When the fluid flow is restricted, the piezoelectric-operated pump may pump less fluid volume per pumping cycle than when the fluid flow is not restricted. Because of this, fewer inflations of the inflatable member may be possible on a single battery charge when the fluid flow is restricted, compared to when the fluid flow is unrestricted. This may be manifested in a change of slope of a graph of the battery voltage as a function of the number of inflations on one battery charge, for example, as shown in FIG. 13A. However, the cause of the change of slope due to a restriction on the fluid flow can be distinguished from a change in slope due to the change in the impedance of the piezoelectric element 1114 by measuring the impedance of the piezoelectric element, for example, as explained with reference to FIG. 14.

The controller 1116 can determine the existence of a fault or failure in a component of the implantable device based on the signals provided by the monitor circuits 1104, 1106, 1110, 1112. For example, the controller 1116 can compare a measured value, or a value derived from a measured value, to a predetermined threshold value, and if the measured value or derived value exceeds the threshold value by more than a predetermined amount or percentage, the controller 1116 can determine that a fault or a failure exists in the implantable device.

In response to a determination of a fault or failure in a component of the implantable device, various actions can be performed by the controller 1116. For example, in one implementation, when a fault or failure is detected that indicates that the implantable device cannot continue to be used safely (e.g., due to a catastrophic short-circuit in a component of the device), the controller 1116 can send a signal to the processor 108A that causes the processor to block the provision of electrical power to the fluid control system 106 and that causes the communication module 108C to communicate a message to the external controller 120, so that the external controller can alert the patient, a physician, or a technician of the fault or failure. The processor can continue to block the provision of electrical power to the fluid control system until a reset signal is received by the processor from the external controller 120.

In another implementation, when a fault or failure is detected that indicates that the performance of a component of the implantable device has degraded but that the implantable device can continue to be used safely, the controller 1116 can send one or more signals to the driver 1108, which can cause the driver to change the waveform that is provided to the piezoelectric element 1114. The changed waveform can optimize the continued performance of the implantable device in view of the degraded performance of one or more components of the device.

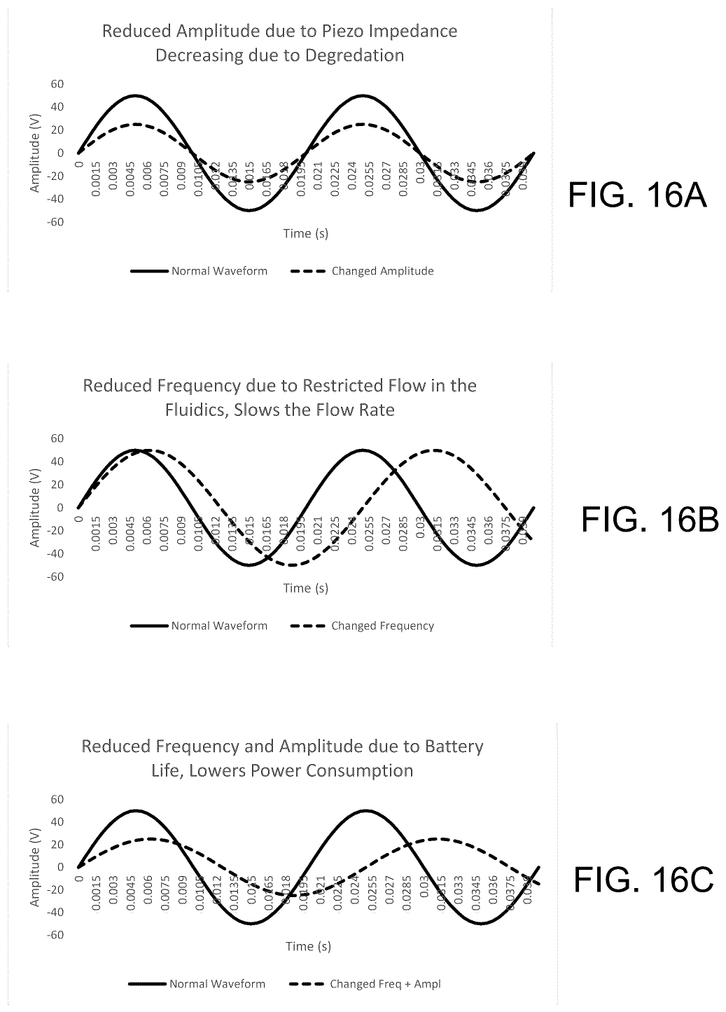

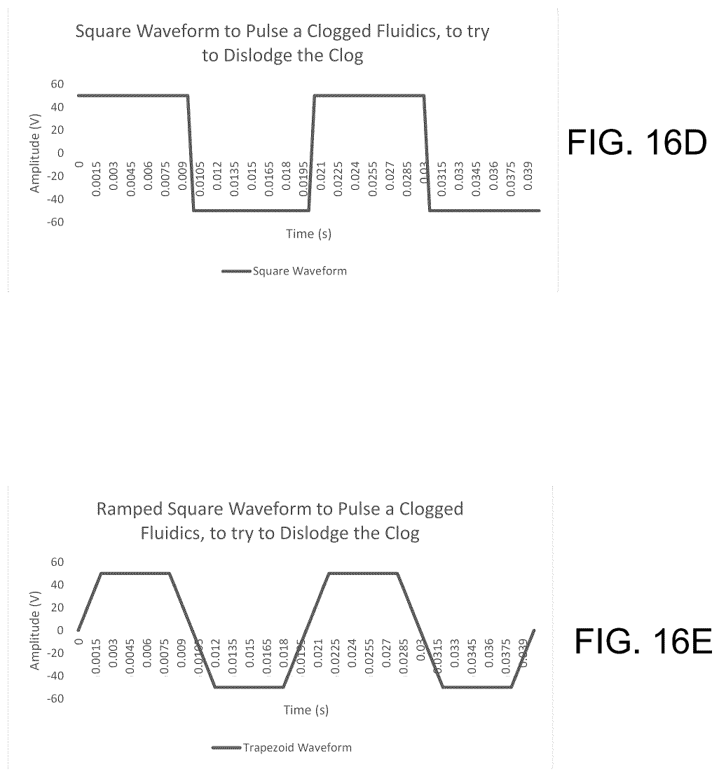

FIG. 16A is a graph of an example normal waveform (solid line) having a peak-to- peak amplitude of about 100 V and a frequency of about 50 Hz that can be provided from the driver 1108 to the piezoelectric element 1114 when the piezoelectric element is in a new, undegraded condition. FIG. 16A also shows a graph of an example changed waveform (dashed line) having a peak-to-peak amplitude of about 50 V and a frequency of about 50 Hz that can be provided from the driver 1108 to the piezoelectric element 1114 when the piezoelectric element is determined to be degraded as a result of the decreased impedance of the element. The decreased amplitude of the changed waveform, as compared to the amplitude of the normal waveform may mitigate the effect of the decreased impedance of the piezoelectric device, which may otherwise result in excessive power consumption by the piezoelectric device and accelerated depletion of the battery 1102.

FIG. 16B is a graph of an example normal waveform (solid line) having a peak-to-peak amplitude of about 100 V and a frequency of about 50 Hz that can be provided from the driver 1108 to the piezoelectric element 1114 when the fluidic components of the implantable device are in a normal operating state. FIG. 16B also shows a graph of an example changed waveform (dashed line) having a peak-to-peak amplitude of about 100 V and a frequency of about 40 Hz that can be provided from the driver 1108 to the piezoelectric element 1114 when the fluidic components of the implantable device are determined to be degraded as a result of a restriction of the fluid flow in the implantable device. The decreased frequency of the changed waveform, as compared to the frequency of the normal waveform may mitigate the effect of the restricted fluid flow in the implantable device, which may otherwise result in inefficient pumping of fluid by the piezoelectric device and accelerated depletion of the battery 1102.

FIG. 16C is a graph of an example normal waveform (solid line) having a peak-to-peak amplitude of about 100 V and a frequency of about 50 Hz that can be provided from the driver 1108 to the piezoelectric element 1114 when the battery 1102 is in a new, undegraded condition. FIG. 16C also shows a graph of an example changed waveform (dashed line) having a peak-to-peak amplitude of about 50 V and a frequency of about 40 Hz that can be provided from the driver 1108 to the piezoelectric element 1114 when the battery 1102 is determined to be degraded. The decreased frequency and amplitude of the changed waveform, as compared to the frequency and amplitude of the normal waveform may assist in prolonging the useful life of the battery 1102 while the battery is used to power the piezoelectric-operated pump.

FIG. 16D is a graph of an example square wave waveform having a peak-to-peak amplitude of about 100 V and a frequency of about 50 Hz that can be provided from the driver 1108 to the piezoelectric element 1114 when a fluidic component of the implantable device is determined to be degraded as a result of a restriction of the fluid flow in the implantable device. The square wave waveform is provided, so that that the piezoelectric-operated pump can provide a sudden change in pressure of the fluidic component to attempt to dislodge a clog and to relieve the fluidic component of the restriction. The square wave of FIG. 16D may consume a relatively large amount of battery power, as compared with the waveforms of FIGS. 16A-16C, but isolated or intermittent use of such a waveform can be advantageous to remove or prevent clogs in fluidic components of the implantable device.

FIG. 16E is a graph of an example trapezoid wave waveform having a peak-to-peak amplitude of about 100 V and a frequency of about 50 Hz that, like the square wave of FIG. 16D, can be provided from the driver 1108 to the piezoelectric element 1114 when a fluidic component of the implantable device is determined to be degraded as a result of a restriction of the fluid flow in the implantable device. The trapezoid wave waveform can be provided to dislodge a clog and to relieve the fluidic component of the restriction. The trapezoid wave, like the square wave of FIG. 16D, may provide a sudden change in the pressure in the fluidic component but with a more gradual change, so as to avoid damage to the piezoelectric device and/or to mechanical or fluidic components of the implantable device.

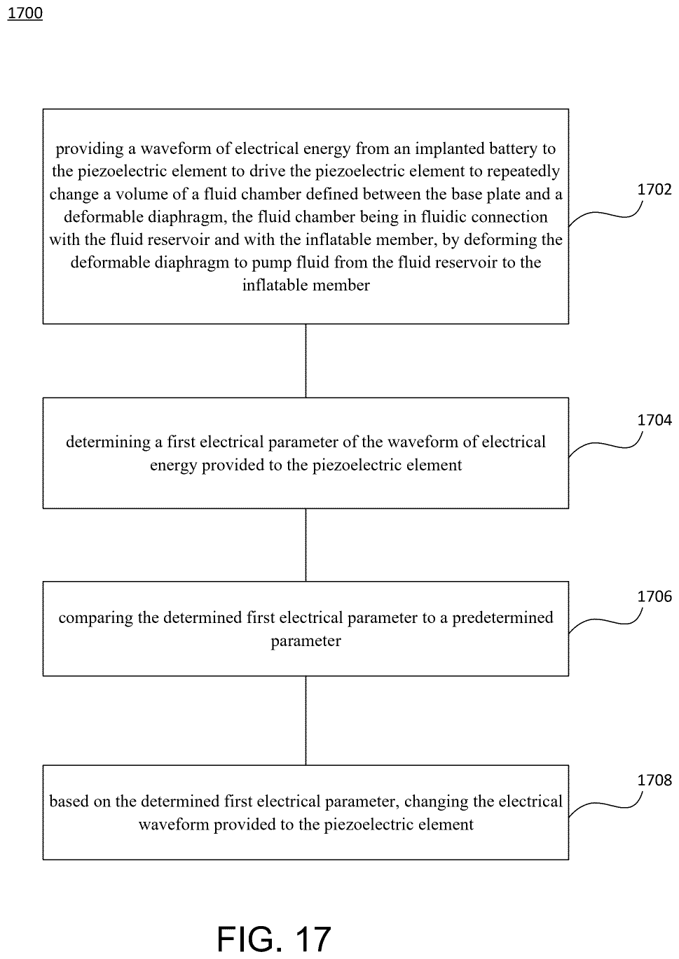

FIG. 17 is a flowchart of an example process 1700 for operating an implanted fluid- operated device having a piezoelectric-operated pump for controlling fluid flow between an implanted fluid reservoir and an implanted inflatable member. The process 1700 includes providing a waveform of electrical energy from an implanted battery to the piezoelectric element to drive the piezoelectric element to repeatedly change a volume of a fluid chamber defined between the base plate and a deformable diaphragm, the fluid chamber being in fluidic connection with the fluid reservoir and with the inflatable member, by deforming the deformable diaphragm to pump fluid from the fluid reservoir to the inflatable member (1702). The process 1700 further includes determining a first electrical parameter of the waveform of electrical energy provided to the piezoelectric element (1704). The process 1700 further includes comparing the determined first electrical parameter to a predetermined parameter (1706). The process 1700 further includes based on the determined first electrical parameter, changing the electrical waveform provided to the piezoelectric element (1708).

While certain features of the described implementations have been illustrated as described herein, many modifications, substitutions, changes and equivalents will now occur to those skilled in the art. It is, therefore, to be understood that the will and in and in appended claims are intended to cover all such modifications and changes as fall within the scope of the embodiments.

What is claimed is:

1. An implantable fluid-operated device configured to control fluid flow between a fluid reservoir and an inflatable member, the device comprising:
a battery configured for storing energy;
a base plate;
a deformable diaphragm;
a fluid chamber defined between the base plate and the deformable diaphragm, the fluid chamber being in fluidic connection with the fluid reservoir and with the inflatable member, wherein the base plate defines a first fluid passageway configured for providing fluid from the fluid reservoir into the fluid chamber and a second fluid passageway configured for providing fluid from the fluid chamber to the inflatable member;
a piezoelectric element attached to the deformable diaphragm;
driver circuitry configured for providing a waveform of electrical energy from the battery to the piezoelectric element to drive the piezoelectric element to repeatedly change a volume of the fluid chamber by deforming the deformable diaphragm to pump fluid from the fluid reservoir to the inflatable member; and
a first monitor circuit configured for determining a first electrical parameter of the waveform of electrical energy provided from the drive circuitry to the piezoelectric element to drive the piezoelectric element; and
a processor configured to, based on the determined first electrical parameter, change the electrical waveform provided to the piezoelectric element.

2. The implantable fluid-operated device of claim 1, wherein the determined first electrical parameter includes a voltage of the waveform of electrical energy provided to the piezoelectric element.

3. The implantable fluid-operated device of claim 2, wherein the processor is configured to compare the determined voltage of the waveform of electrical energy provided to the piezoelectric element to a predetermined threshold voltage and, when the determined voltage exceeds the predetermined voltage, to change the electrical waveform provided to the piezoelectric element.

4. The implantable fluid-operated device of claim 1, wherein the determined first electrical parameter includes a rate of change of a voltage of the waveform of electrical energy provided to the piezoelectric element.

5. The implantable fluid-operated device of claim 4, wherein the processor is configured to compare the determined rate of change of the voltage of the waveform of electrical energy provided to the piezoelectric element to a predetermined threshold change of the voltage and, when the rate of change of the determined voltage exceeds the predetermined voltage, to change the electrical waveform provided to the piezoelectric element.

6. The implantable fluid-operated device of claim 1, wherein changing the electrical waveform provided to the piezoelectric element includes one or more of changing an amplitude of the waveform, changing a frequency of the waveform, and changing a rate of change of a voltage of the waveform with respect to time.

7. The implantable fluid-operated device of claim 1, wherein changing the electrical waveform provided to the piezoelectric element includes blocking the providing of the waveform to the piezoelectric element until a reset signal is received by the processor.

8. The implantable fluid-operated device of claim 1, wherein the determined first electrical parameter includes a current of the waveform of electrical energy provided to the piezoelectric element.

9. The implantable fluid-operated device of claim 8, wherein the processor is configured to compare the determined current of the waveform of electrical energy provided to the piezoelectric element to a predetermined threshold and, when the determined current exceeds the predetermined current, to change the electrical waveform provided to the piezoelectric element.

10. The implantable fluid-operated device of claim 1, further comprising a second monitor circuit configured for determining a second electrical parameter of electrical energy provided from the battery to the driver circuitry and wherein the processor is further configured to, based on the determined second electrical parameter, change the electrical waveform provided to the piezoelectric element.

11. The implantable fluid-operated device of claim 1, further comprising a fluid filter disposed within or at an end of the first fluid passageway or disposed within or at an end of the second fluid passageway, wherein the end of the first fluid passageway is distal from the fluid chamber and wherein the end of the second fluid passageway is distal from the fluid chamber.

12. The implantable fluid-operated device of claim 1, further comprising power transmission circuitry configured for receiving power from an external power source and providing power to charge the battery.

13. A method of operating an implanted fluid-operated device having a pump operated by a piezoelectric element for controlling fluid flow between an implanted fluid reservoir and an implanted inflatable member, the method comprising:
providing a waveform of electrical energy from an implanted battery to the piezoelectric element to drive the piezoelectric element to repeatedly change a volume of a fluid chamber defined between a base plate and a deformable diaphragm, the fluid chamber being in fluidic connection with the fluid reservoir and with the inflatable member, by deforming the deformable diaphragm to pump fluid from the fluid reservoir to the inflatable member;
determining a first electrical parameter of the waveform of electrical energy provided to the piezoelectric element;
comparing the determined first electrical parameter to a predetermined parameter; and
based on the determined first electrical parameter, changing the electrical waveform provided to the piezoelectric element.

14. The method of claim 13, wherein the determined first electrical parameter includes a voltage of the waveform of electrical energy provided to the piezoelectric element, the method further comprising comparing the determined voltage of the waveform of electrical energy to a predetermined threshold voltage and, when the determined voltage exceeds the predetermined voltage, changing the electrical waveform provided to the piezoelectric element.

15. The method of claim 13, wherein the determined first electrical parameter includes a rate of change of a voltage of the waveform of electrical energy provided to the piezoelectric element, the method further comprising comparing the determined rate of change of the voltage of the waveform of electrical energy provided to the piezoelectric element to a predetermined threshold change of the voltage and, when the rate of change of the determined voltage exceeds the predetermined voltage, changing the electrical waveform provided to the piezoelectric element.

16. The method of claim 13, wherein changing the electrical waveform provided to the piezoelectric element includes one or more of changing an amplitude of the waveform, changing a frequency of the waveform, and changing a rate of change of a voltage of the waveform with respect to time.

17. The method of claim 13, wherein changing the electrical waveform provided to the piezoelectric element includes blocking the providing of the waveform to the piezoelectric element until a reset signal is received.

18. The method of claim 13, wherein the determined first electrical parameter includes a current of the waveform of electrical energy provided to the piezoelectric element.

19. The method of claim 18, further comprising comparing the determined current of the waveform of electrical energy provided to the piezoelectric element to a predetermined threshold and, when the determined current exceeds the predetermined current, changing the electrical waveform provided to the piezoelectric element.

20. The method of claim 13, further comprising:
determining a second electrical parameter of electrical energy provided from the battery; and
based on the determined second electrical parameter, changing the electrical waveform provided to the piezoelectric element.
